(19) United States
(12) Patent Application Publication
(10) Pub. No.: US 2025/0057764 A1
(43) Pub. Date: Feb. 20, 2025

(54) COMPOSITIONS, METHODS AND SYSTEMS FOR AEROSOL DRUG DELIVERY

(71) Applicant: ASTRAZENECA AB, Sodertalje (SE)

(72) Inventors: Kellisa Beth LACHACZ, Wilmington, DE (US); Vidya B. JOSHI, Wilmington, DE (US); James ARCHBELL, Wilmington, DE (US); David LECHUGA-BALLESTEROS, Wilmington, DE (US); Michael RIEBE, Wilmington, DE (US)

(21) Appl. No.: 18/719,343

(22) PCT Filed: Dec. 16, 2022
(86) PCT No.: PCT/IB2022/062387

§ 371 Date: Jun. 13, 2024

Related U.S. Application Data
(60) Provisional application No. 63/291,538, filed on Dec. 20, 2021.

(51) Int. Cl. A61K 9/12; A61K 9/00; A61K 31/137; A61K 31/167; A61K 31/40; A61K 31/44; A61K 31/58; A61K 47/06; A61K 47/24

(52) U.S. Cl. CPC A61K 9/124; A61K 9/008; A61K 31/137; A61K 31/167; A61K 31/40; A61K 31/44; A61K 31/58; A61K 47/06; A61K 47/24

(57) ABSTRACT

1. A pharmaceutical composition deliverable from a metered dose inhaler, the pharmaceutical composition comprising: a propellant of pharmaceutical grade 1,1-Difluoroethane (HFC-152a); a plurality of active agent particles; and a plurality of phospholipid particles comprising perforated microstructures; wherein the active agent particles comprise an active agent selected from a long-acting muscarinic antagonist (LAMA), a long-acting P2-agonist (LABA), a short-acting beta-agonist (SABA), an inhaled corticosteroid (ICS), and a non-corticosteroid anti-inflammatory agent. A metered dose inhaler comprising a canister with an outlet valve including an actuator for dispensing a metered amount of said pharmaceutical composition, wherein the canister contains the pharmaceutical composition. Said composition for use in the treatment of a pulmonary disease or disorder.

### CROSS-REFERENCE TO RELATED APPLICATIONS

This application claims the benefit of priority to U.S. Provisional Application No. 63/291,538, filed on Dec. 20, 2021, which is incorporated by reference herein in its entirety for all purpose.

### BACKGROUND

Methods of targeted drug delivery that deliver an active agent at the site of action are often desirable. For example, targeted delivery of active agents can reduce undesirable side effects, lower dosing requirements, and decrease therapeutic costs. In the context of respiratory delivery, inhalers are well known devices for administering an active agent to a subject's respiratory tract, and several different inhaler systems are currently commercially available. Three common inhaler systems include dry powder inhalers, nebulizers, and metered dose inhalers (MDIs), also known as pressurized metered dose inhalers (pMDIs).

MDIs may be used to deliver medicaments in a solubilized form or as a suspension. Typically, MDIs use a relatively high vapor pressure propellant to expel aerosolized droplets containing an active agent into the respiratory tract when the MDI is activated. Dry powder inhalers generally rely on the patient's inspiratory efforts to introduce a medicament in a dry powder form to the respiratory tract. Nebulizers form a medicament aerosol to be inhaled by imparting energy to a liquid solution or suspension.

MDIs are active delivery devices that utilize the pressure generated by a propellant. The propellant must be safe for patients' use and be pharmaceutically acceptable. The active agent to be delivered by an MDI is typically provided as a suspension of fine particulates dispersed within a propellant or a combination of two or more propellants (i.e., a propellant “system”). However, fine particles of active agent suspended in a propellant or propellant system tend to aggregate or flocculate rapidly. In turn, aggregation or flocculation of these fine particles may complicate the delivery of the active agent. Another problem associated with such suspension MDI formulations relates to crystal growth of the drug during storage, resulting in a decrease over time of aerosol properties and delivered dose uniformity of such MDIs. Thus, it is critical to properly formulate the active agents with the excipients and propellants to form a stable suspension suitable for MDI. The properties of the propellant play an important role in the performance of a suspension formulation for MDIs. For example, the liquid density, vapor pressure and water solubility of a propellant affect the suspension stability, dose uniformity, aerosol performance and moisture ingress. Other properties of a propellant, such as dipole moment, surface tension, boiling point, liquid viscosity, latent heat, etc., are also factors to be considered when formulating the suspension formulation. Thus, there remains a need to research and develop innovative suspension MDI formulations with the desired characteristics.

### BRIEF SUMMARY

The present disclosure provides compositions, methods, and systems for respiratory delivery of one or more active agents.

In some embodiments, the compositions described herein are formulated for pulmonary delivery of one or more active agents via an MDI. In other embodiments, the compositions described herein may be formulated for nasal delivery via an MDI. In some embodiments, the compositions comprise a propellant of pharmaceutical grade 1,1-difluoroethane (HFC-152a), a plurality of active agent particles, and a plurality of phospholipid particles comprising perforated microstructures. In some embodiments, the plurality of active agent particles comprise one, two, three or four active agents selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent.

In some embodiments, the compositions comprise a propellant of pharmaceutical grade 1,1-difluoroethane (HFC-152a), a plurality of LAMA particles, and a plurality of phospholipid particles comprising perforated microstructures. In some embodiments, the compositions comprise a propellant of pharmaceutical grade 1,1-difluoroethane (HFC-152a), a plurality of LABA particles, and a plurality of phospholipid particles comprising perforated microstructures. In some embodiments, the compositions comprise a propellant of pharmaceutical grade 1,1-difluoroethane (HFC-152a), a plurality of SABA particles, and a plurality of phospholipid particles comprising perforated microstructures. In some embodiments, the compositions comprise a propellant of pharmaceutical grade 1,1-difluoroethane (HFC-152a), a plurality of ICS particles, and a plurality of phospholipid particles comprising perforated microstructures. In some embodiments, the compositions comprise a propellant of pharmaceutical grade 1,1-difluoroethane (HFC-152a), a plurality of non-corticosteroid anti-inflammatory agent particles, and a plurality of phospholipid particles comprising perforated microstructures.

In some embodiments, the compositions comprise a propellant of pharmaceutical grade 1,1-difluoroethane (HFC-152a), a plurality of active agent particles and a plurality of phospholipid particles comprising perforated microstructures. In some embodiments, the compositions comprise a propellant of pharmaceutical grade 1,1-difluoroethane (HFC-152a), a plurality of a first species of active agent particle, a plurality of a second species of active agent particle, and a plurality of phospholipid particles comprising perforated microstructures. In some embodiments, the first species of active agent particles comprise a first active agent and the second species of active agent particles comprise a second active agent. In some embodiments, the compositions described herein further comprise a plurality of a third species of active agent particle, wherein the third species of active agent particle comprises a third active agent. In some embodiments, the compositions described herein further comprise a plurality of a fourth species of active agent particle, wherein the fourth species of active agent particle comprises a fourth active agent. In some embodiments, the active agents are selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent. In some embodiments, the first and second active agents are selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent. In further embodiments, the third active agent is selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent. In yet further embodiments, the fourth active agent is selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent.

The methods described herein include methods of treating a pulmonary disease or disorder in a patient by actuating a metered dose inhaler containing a composition as described herein.

Also described herein are systems for pulmonary delivery of one or more active agents. In some embodiments, such systems include an MDI comprising a canister with an outlet valve including an actuator (e.g., a depressible valve stem) for dispensing a metered amount of a composition as described herein.

### BRIEF DESCRIPTION OF THE SEVERAL VIEWS OF THE DRAWINGS

FIG. 1 shows budesonide (BD), glycopyrrolate (GP), and formoterol fumarate (FF), Aerodynamic Particle Size Distribution by NGI of BGF-152a.

FIG. 2 shows BD Aerodynamic Particle Size Distribution by NGI Stability Data for BGF-152a, 25° C./60% RH—Valve Down, Protected at initial, 1 month, 3 months, 6 months, and 12 months.

FIG. 3 shows GP Aerodynamic Particle Size Distribution by NGI Stability Data for BGF-152a, 25° C./60% RH—Valve Down, Protected at initial, 1 month, 3 months, 6 months, and 12 months.

FIG. 4 shows FF Aerodynamic Particle Size Distribution by NGI Stability Data for BGF-152a, 25° C./60% RH—Valve Down, Protected at initial, 1 month, 3 months, 6 months, and 12 months.

FIG. 5 shows BD Aerodynamic Particle Size Distribution by NGI Stability Data for BGF-152a, 40° C./75% RH—Valve Down, Protected at initial, 1 month, 3 months, and 6 months.

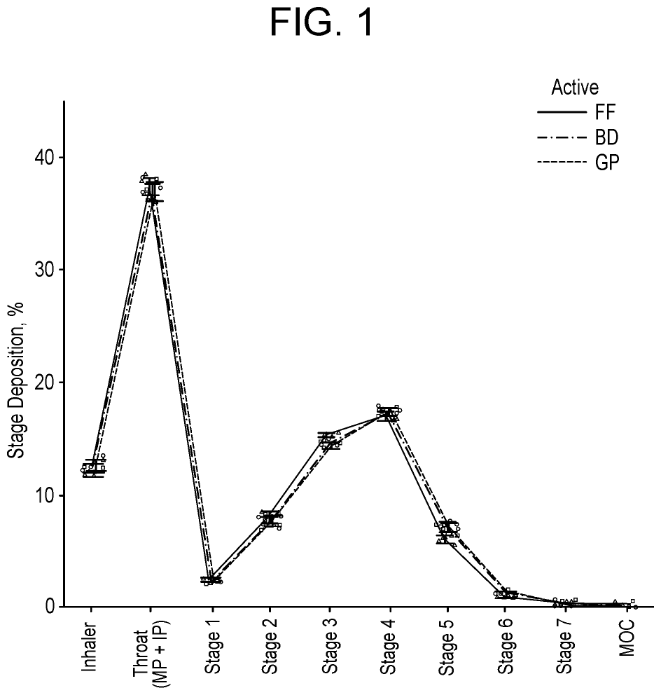

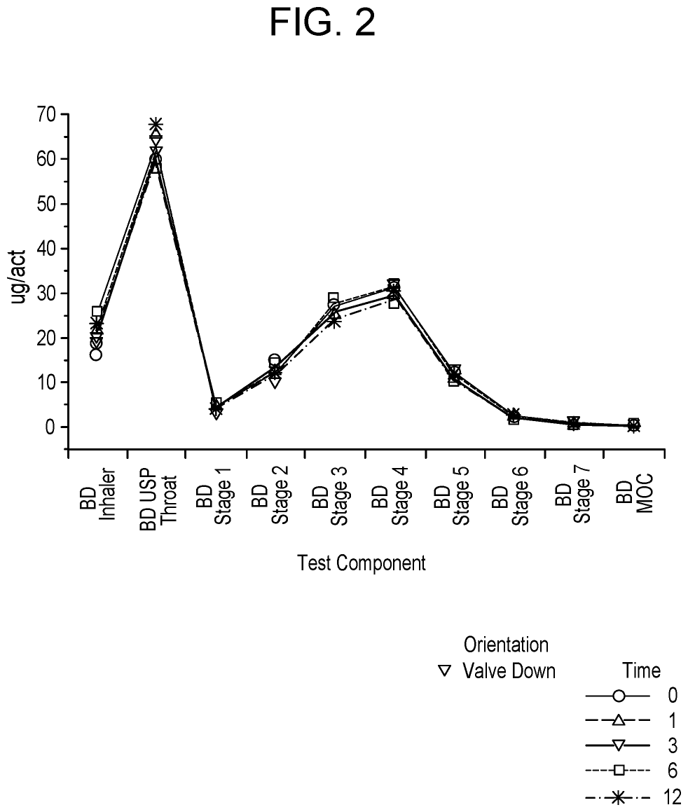

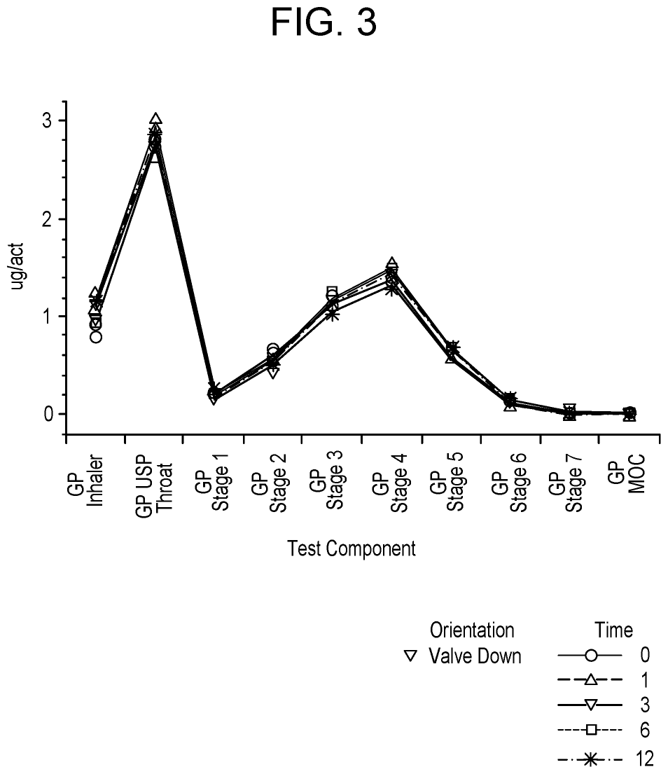

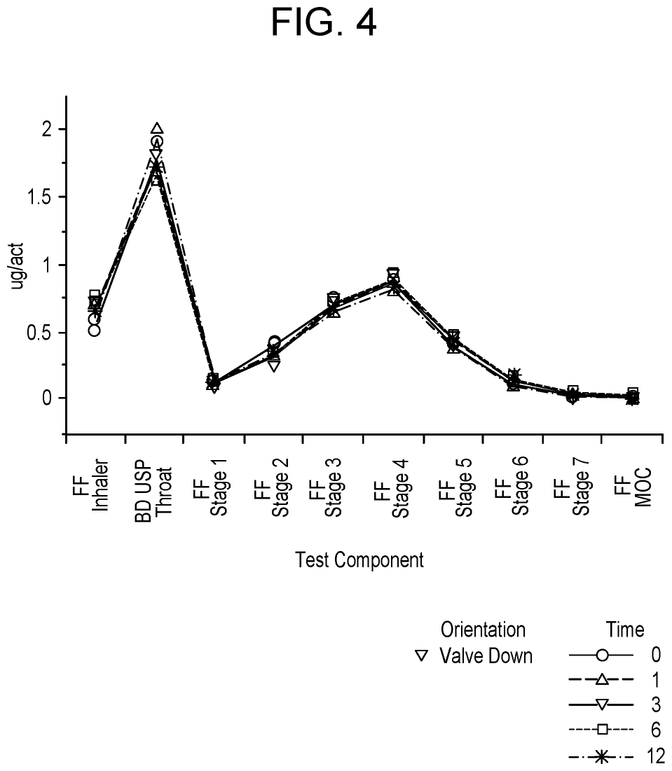

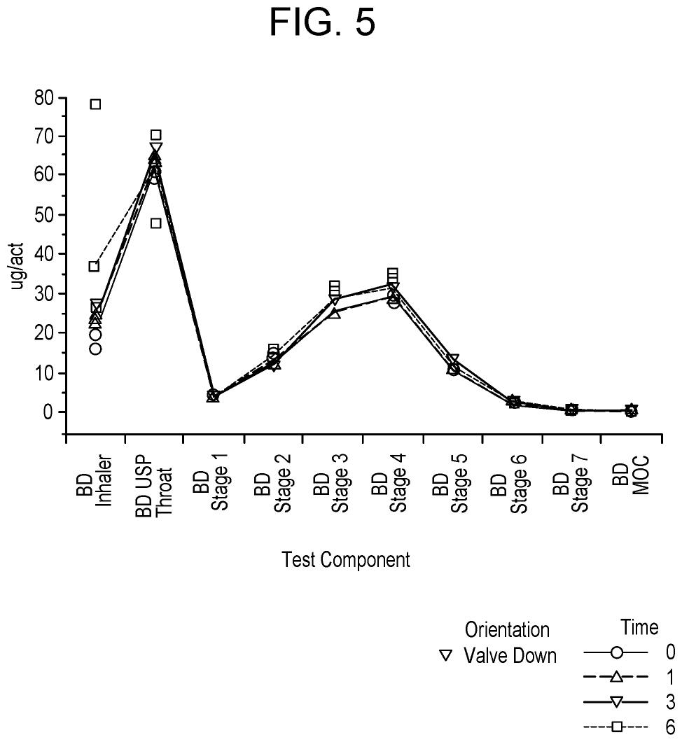

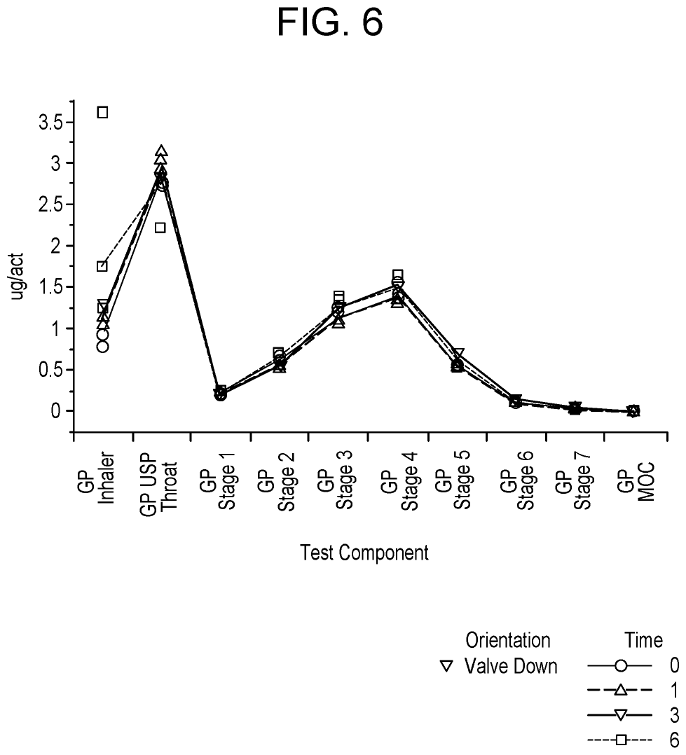

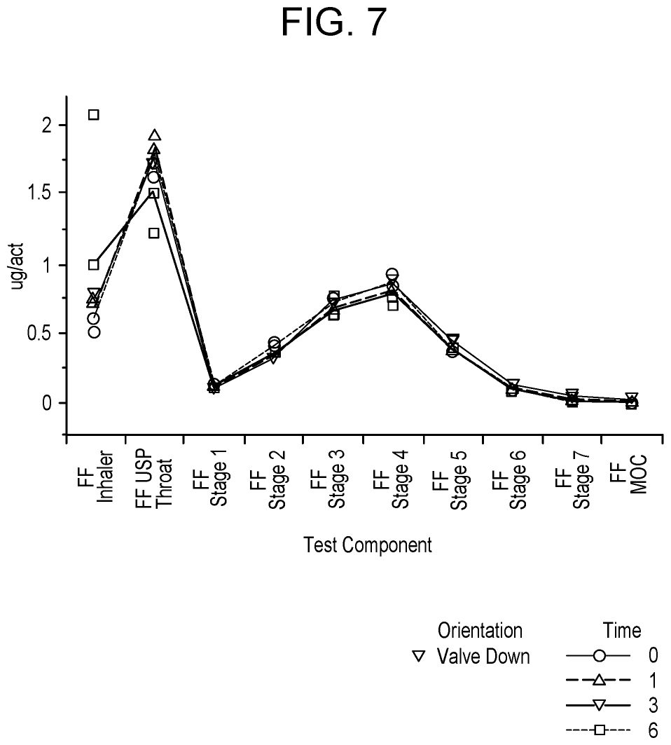

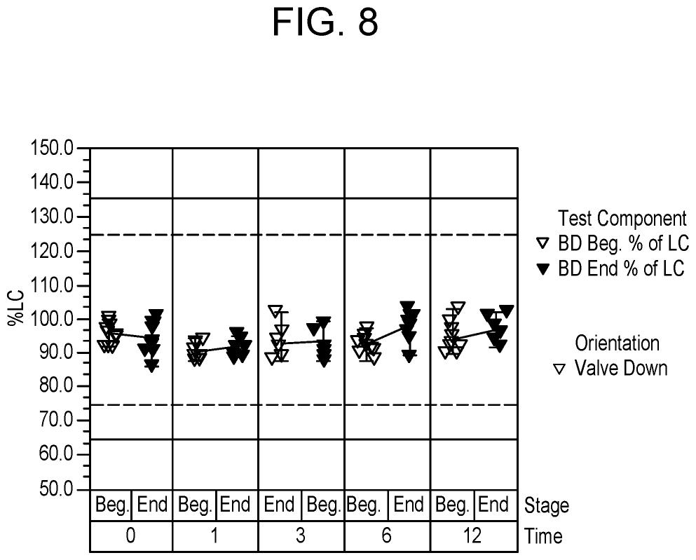

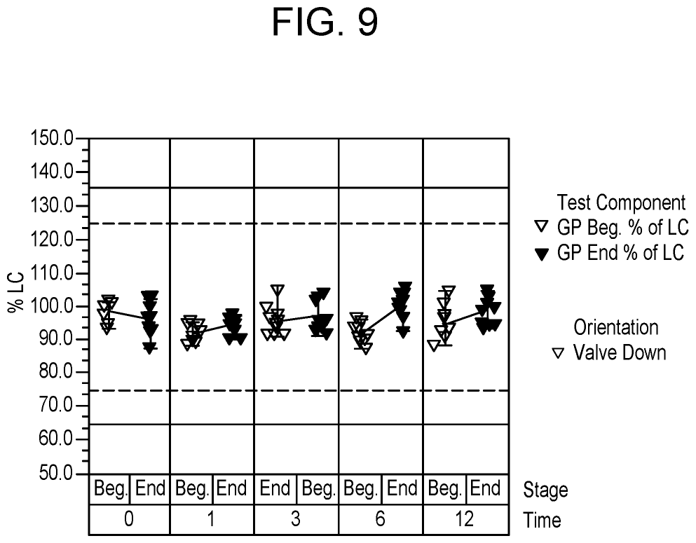

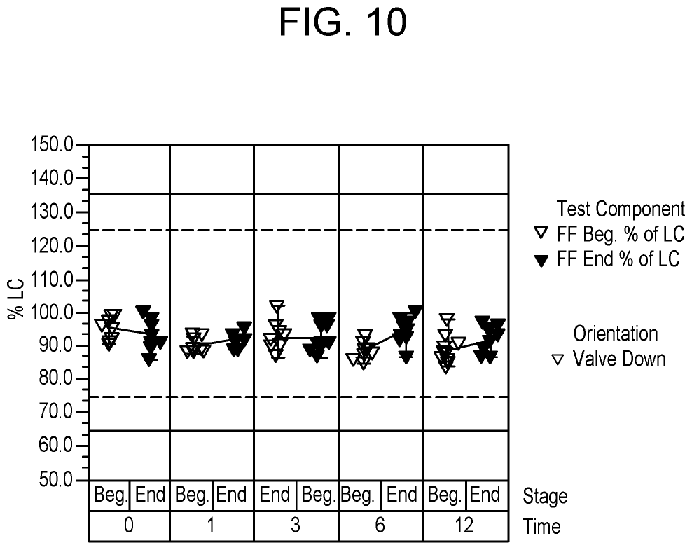

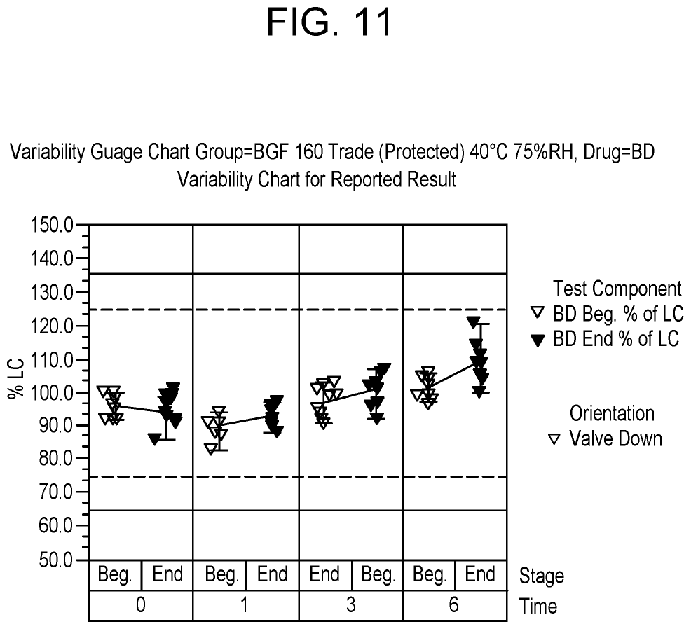

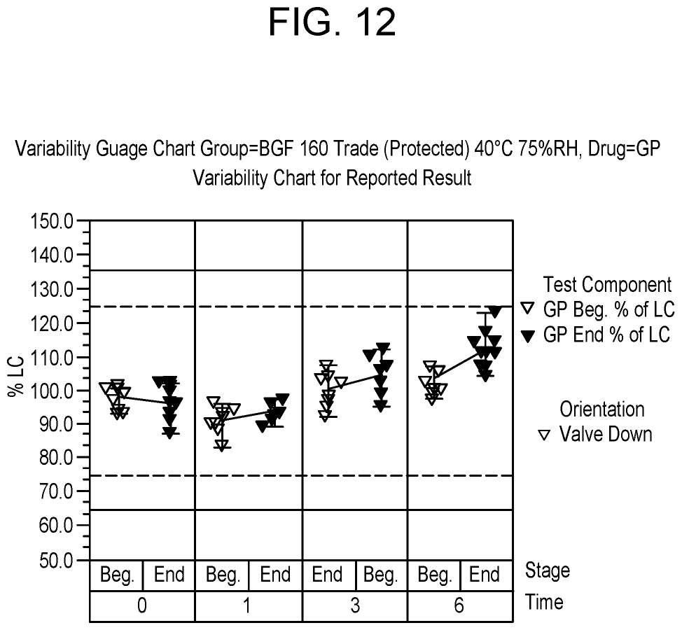

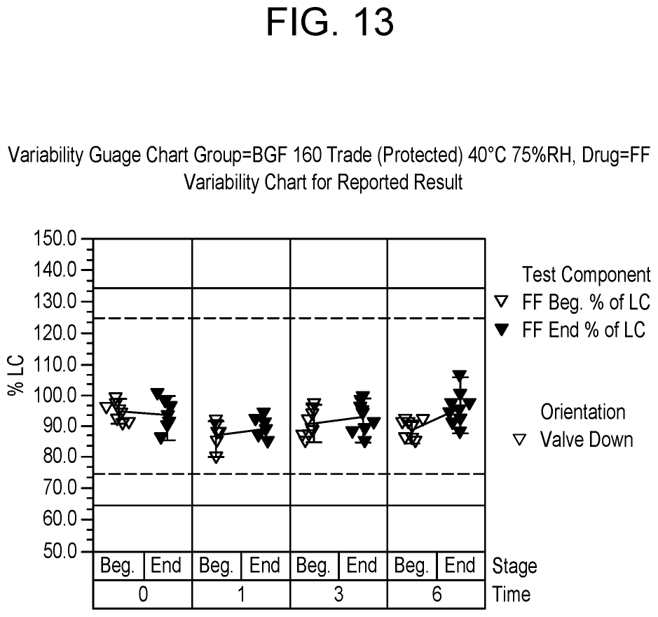

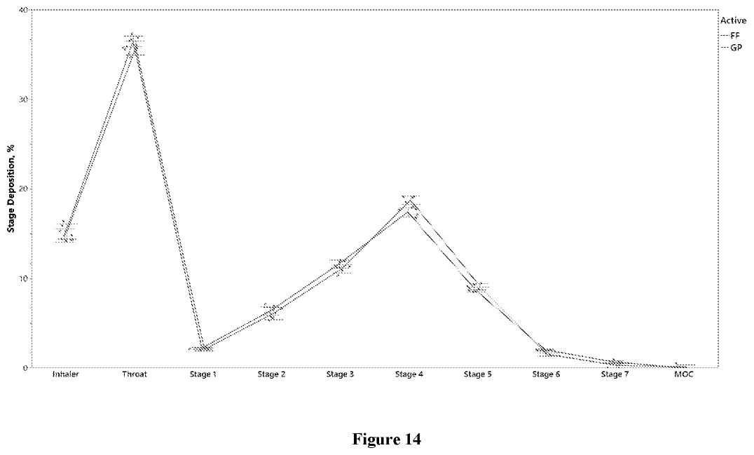

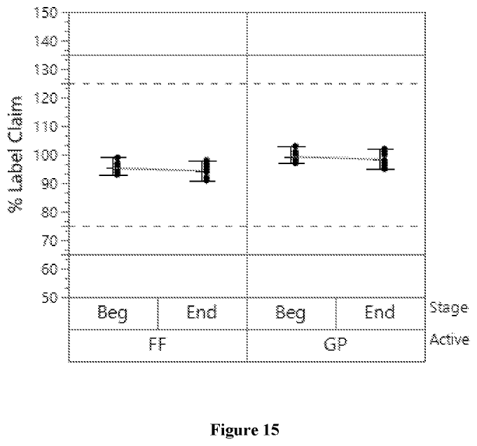

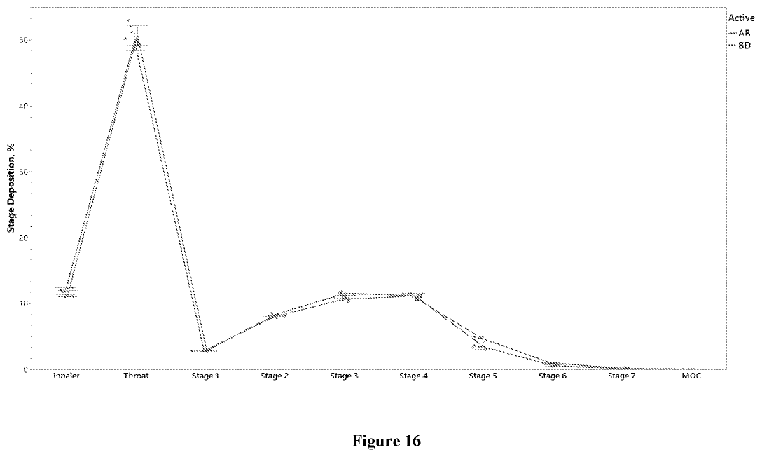

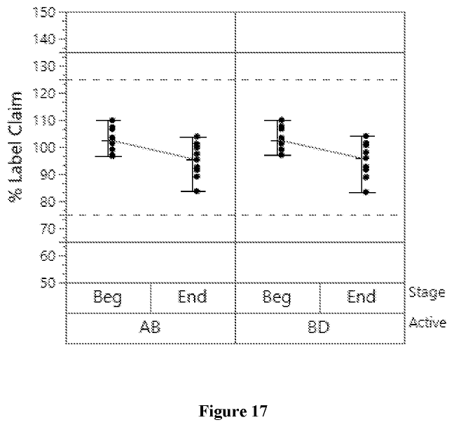

FIG. 1 shows GP Aerodynamic Particle Size Distribution by NGI Stability Data for BGF-152a, 40° C./75% RH—Valve Down, Protected at initial, 1 month, 3 months, and 6 months.

FIG. 7 shows FF Aerodynamic Particle Size Distribution by NGI Stability Data for BGF-152a, 40° C./75% RH—Valve Down, Protected at initial, 1 month, 3 months, and 6 months.

FIG. 8 shows BD Delivered Dose Uniformity Stability Data for BFF-152a, 25° C./60% RH—Valve Down, Protected.

FIG. 9 shows GP Delivered Dose Uniformity Stability Data for BFF-152a, 25° C./60% RH—Valve Down, Protected.

FIG. 10 shows FF Delivered Dose Uniformity Stability Data for BFF-152a, 25° C./60% RH—Valve Down, Protected.

FIG. 11 shows BD Delivered Dose Uniformity Stability Data for BFF-152a, 40° C./75% RH—Valve Down, Protected.

FIG. 12 shows GP Delivered Dose Uniformity Stability Data for BFF-152a, 40° C./75% RH—Valve Down, Protected.

FIG. 13 shows FF Delivered Dose Uniformity Stability Data for BFF-152a, 40° C./75% RH—Valve Down, Protected.

FIG. 14 shows GP and FF, Aerodynamic Particle Size Distribution by NGI of GFF-152a.

FIG. 15 shows GP and FF Delivered Dose Uniformity of GFF-152a.

FIG. 16 shows BD and AB (albuterol sulfate), Aerodynamic Particle Size Distribution by NGI of BDA-152a.

FIG. 17 shows BD and AB Delivered Dose Uniformity of BDA-152a.

### DETAILED DESCRIPTION

### Definitions

Unless specifically defined otherwise, the technical terms, as used herein, have their normal meaning as understood in the art. The following terms are specifically defined for the sake of clarity.

The term “active agent” is used herein to include any agent, drug, compound, composition, or other substance that may be used on, or administered to, a human or animal for any purpose, including therapeutic, pharmaceutical, pharmacological, diagnostic, cosmetic, and prophylactic agents and immunomodulators. Active agent may be used interchangeably with the terms drug, pharmaceutical, medicament, drug substance, or therapeutic. As used herein, active agent may also encompass natural or homeopathic products that are not generally considered therapeutic.

The terms “associate,” “associate with,” or “association” refer to an interaction or relationship between a chemical entity, composition, or structure in a condition of proximity of a surface, such as the surface of another chemical entity, composition, or structure. Association includes, for example, adsorption, adhesion, covalent bonding, hydrogen boding, ionic bonding and electrostatic attraction, Lifshitz-van der Waals interactions, and polar interactions. The terms “adhere” or “adhesion” refer to a form of association, and are used a generic terms for all forces tending to cause a particle or mass to be attracted to a surface. Adhere also refers to bringing and keeping particles in contact with each other, such that there is substantially no visible separation between particles due to their different buoyancies in a propellant under normal conditions. In one embodiment, a particle that attaches to or binds to a surface is encompassed by the term adhere. Normal conditions may include storage at room temperature or under an accelerative force due to gravity. As described herein, active particles may associate with suspending particles to form a co-suspension, where there is substantially no visible separation between the suspending particles and the active agent particles or flocculates thereof due to differences in buoyance within a propellant.

“Suspending particles” refer to a material or combination of materials that is acceptable for respiratory delivery and acts as a vehicle for active agent particles. Suspending particles interact with the active agent particle to facilitate repeatable dosing, delivery, or transport of active agent to the target site of delivery, i.e., the respiratory tract. The suspending particles described herein are dispersed within a suspension medium including a propellant or propellant system, and can be configured according to any shape, size, or surface characteristic suited to achieving a desired suspension stability or active agent delivery performance. Exemplary suspending particles include particles that exhibit a particle size that facilitates respiratory delivery of active agent and have physical configurations suited to formulation and delivery of the stabilized suspensions as described herein.

The term “co-suspension” refers to a suspension of two or more types of particles having different compositions within a suspension medium, wherein one type of particle associates at least partially with one or more of the other particle types. The association leads to an observable change in one or more characteristics of at least one of the individual particle types suspended in the suspension medium. Characteristics modified by the association may include, for example, one or more of the rate of aggregation or flocculation, the rate and nature of separation, i.e., sedimentation or creaming, density of a cream or sediment layer, adhesion to container walls, adhesion to valve components, and the rate and level of dispersion upon agitation. The term co-suspension includes partial co-suspensions, where a majority of the at least two particle types associate with each other, however, some separation (i.e., less than a majority) of the at least two particle types may be observed.

The term “metered dose” refers to the amount of active agent contained in the volume of formulation that exits the canister upon actuation of an MDI. The term “delivered dose” refers to the amount of active agent contained in the volume of formulation that exits the actuator nozzle and is available to be drawn into a patient's lungs.

In the context of a composition containing or providing respirable aggregates, particles, drops, etc., such as compositions described herein, the term “fine particle dose” or “FPD” refers to the dose, either in total mass or fraction of the nominal dose or metered dose, that is within a respirable range. The dose that is within the respirable range in measured in vitro to be the sum of the dose delivered at stages 3 through Micro Orifice Collector in a Next Generation Impactor operated at a flow rate of 30 l/min.

In the context of a composition containing or providing respirable aggregates, particles, drops, etc., such as compositions described herein, the term “fine particle fraction” or “FPF” refers to the proportion of the delivered material relative to the delivered dose (i.e. the amount that exits the actuator of a delivery device, such as an MDI) that is within a respirable range. The amount of delivered material within the respirable range is measured in vitro as the sum of the material delivered at stages 3 through Micro Orifice Collector in a Next Generation Impactor operated at a flow rate of 30 l/min.

As used herein, the term “inhibit” refers to a measurable lessening of the tendency of a phenomenon, symptom, or condition to occur or the degree to which that phenomenon, symptom, or condition occurs. The term “inhibit”, or any form thereof, is used in its broadest sense and includes minimize, prevent, reduce, repress, suppress, curb, constrain, restrict, slow progress of, and the like.

“Mass median aerodynamic diameter” or “MMAD” as used herein refers to the aerodynamic diameter of an aerosol below which 50% of the mass of the aerosol consists of particles with an aerodynamic diameter smaller than the MMAD, with the MMAD being calculated according to monograph 601 of the United States Pharmacopeia (“USP).

When referred to herein, the term “optical diameter” indicates the size of a particle as measured by the Fraunhofer diffraction mode using a laser diffraction particle size analyzer equipped with a dry powder dispenser (e.g., Sympatec GmbH, Clasthal-Zellerfeld, Germany).

The term “solution mediated transformation” refers to the phenomenon in which a more soluble form of a solid material (i.e., particles with small radius of curvature (a driving force for Ostwald ripening), or amorphous material) dissolves and recrystallizes into the more stable crystal form that can coexist in equilibrium with its saturated propellant solution.

A “patient” refers to an animal in which the one or more active agents as described herein will have a therapeutic effect. In some embodiments, the patient is a human being.

“Perforated microstructures” refers to suspending particles that include a structural matrix that exhibits, defines, or comprises voids, pores, defects, hollows, spaces, interstitial spaces, apertures, perforations, or holes that allow the surrounding suspension medium to permeate, fill, or pervade the microstructure, such as those materials and preparations described in U.S. Pat. No. 6,309,623 to Weers, et al., which methods are incorporated herein by reference, and in U.S. Pat. Nos. 8,815,258, 9,463,161, and U.S. Patent Application Publication 2011/0135737. The primary form of the perforated microstructure is, generally, not essential, and any overall configuration that provides the desired formulation characteristics is contemplated herein. Accordingly, in some embodiments, the perforated microstructures may comprise approximately spherical shapes, such as hollow, porous, spray-dried microspheres. However, collapsed, corrugated, deformed, or fractured particulates of any primary form or aspect ratio may also be compatible.

As is true of the suspending particles described herein, perforated microstructures may be formed of any biocompatible material that does not substantially degrade or dissolve in the selected suspension medium. While a wide variety of materials may be used to form the particles, in some embodiments, the structural matrix is associated with, or includes, a surfactant such as a phospholipid or fluorinated surfactant.

The term “suspension medium” as used herein refers to a substance providing a continuous phase within which active agent particles and suspending particles can be dispersed to provide a co-suspension formulation. The suspension medium used in formulations described herein includes propellant. As used herein, the term “propellant” refers to one or more pharmacologically inert substances which exert a sufficiently high vapor pressure at normal room temperature to propel a medicament from the canister of an MDI to a patient on actuation of the MDI's metering valve. Therefore, the term “propellant” refers to both a single propellant and to a combination of two or more different propellants forming a “propellant system.”

The term “respirable” generally refers to particles, aggregates, drops, etc. sized such that they can be inhaled and reach the airways of the lung.

When used to refer to compositions described herein, the terms “physical stability” and “physically stable” refer to a composition that is resistant to one or more of aggregation, flocculation, and particle size changes due to solution mediated transformations and is capable of substantially maintaining the MMAD of suspending particles and the fine particle dose. In some embodiments, physical stability may be evaluated through subjecting compositions to accelerated degradation conditions, such as by temperature cycling.

When referring to active agents, the term “potent” indicates active agents that are therapeutically effective at or below doses ranging from about 0.01 mg/kg to about 1 mg/kg. Typical doses of potent active agents generally range from about 100 μg to about 100 mg.

When referring to active agents, the term “highly potent” indicates active agents that are therapeutically effective at or below doses of about 10 μg/kg. Typical doses of highly potent active agents generally range up to about 100 μg.

The terms “suspension stability” and “stable suspension” refer to suspension formulations capable of maintaining the properties of a co-suspension of active agent particles and suspending particles over a period of time. In some embodiments, suspension stability may be measured through delivered dose uniformity achieved by compositions described herein.

The term “substantially insoluble” means that a composition is either totally insoluble in a particular solvent or it is poorly soluble in that particular solvent. Substantially insoluble means that a particular solute has a solubility of less than one part per 100 parts solvent. The term substantially insoluble includes the definitions of “slightly soluble” (from 100 to 1000 parts solvent per one part solute), “very slightly soluble” (from 1000 to 10,000 parts solvent per one part solute), and “practically insoluble” (more than 10,000 parts solvent per one part solute) as given in Table 16-1 of Remington: The Science and Practice of Pharmacy, 21^{st} ed. Lippincott, Williams & Wilkins, 2006, p. 212.

The term “surfactant” as used herein refers to any agent with preferentially adsorbs to an interface between two immiscible phases, such as the interface between water and an organic polymer solution, a water/air interface, or an organic solvent/air interface. Surfactants generally possess a hydrophilic moiety and a lipophilic moiety, such that upon adsorbing to microparticles they tend to present moieties to the continuous phase that do not attract similarly-coated particles, thus reducing particle agglomeration.

A “therapeutically effective amount” is the amount of compound which achieves a therapeutic effect by inhibiting a disease or disorder in a patient or by prophylactically inhibiting or preventing the onset of a disease or disorder. A therapeutically effective amount may be an amount which relieves to some extent one or more symptoms of a disease or disorder in a patient; returns to normal either partially or completely one or more physiological or biochemical parameters associated with or causative of the disease or disorder; and/or reduces the likelihood of the onset of the disease or disorder.

The terms “chemically stable” and “chemical stability” refer to formulations wherein the individual degradation products of active agent remain below the limits specified by regulatory requirements during the shelf life of the product for human use (e.g., 1% of total chromatographic peak area per ICH guidance Q3B(R2)) and there is acceptable mass balance (e.g. as defined in ICH guidance Q1E) between active agent assay and total degradation products.

### Compositions

The compositions described herein comprise a suspension medium including a propellant, active agent particles, and suspending particles. If desired, the compositions described herein may include one or more additional constituents. Moreover, variations and combinations of components of the compositions described herein may be used. For example, the active agent particles included in the compositions may include two or more active agents, or two or more different species of active agent particles may be used, with each different species of active agent particle including one or more different active gents. Alternatively, two or more species of suspending particles may be used in compositions for the delivery of one or more active agents or active agent particles. In some embodiments, when two or more active agent particles are present, the present composition is in form of a fixed dose combination. By “fixed dose combination”, it is meant two or more active agents in a single dose form, such as a formulation in a single metered dose inhaler.

Generally, due to density differences between distinct species of particles and the medium within which they are suspended (e.g., a propellant or propellant system), buoyancy forces cause creaming of particles with lower density than the propellant and sedimentation of particles with higher density than the propellant. Therefore, in suspensions that consist of a mixture of different types of particles with different density or different tendencies to flocculate, sedimentation or creaming behavior is expected to be specific to each of the different particle types and to the specific suspension medium used, and is expected to lead to separation of the different particle types within the suspension medium.

However, the combinations of propellant, active agent particles, and suspending particles described herein provide co-suspensions wherein active agent particles and suspending particles co-locate within the propellant (i.e., the active agent particles associate with the suspending particles such that suspending particles and active agent particles do not exhibit substantial separation relative to each other, such as by differential sedimentation or creaming, even after a time sufficient for the formation of a cream or sediment layer). In particular, the active agent particles associate with the suspending particles such that there is no substantial separation of active agent particles and suspending particles within the continuous phase formed by the suspension medium under typical patient use conditions.

Compositions of propellant, active agent particles, and suspending particles according to the present description provide desirable chemical stability, suspension stability, and active agent delivery characteristics. For example, in certain embodiments, when present within an MDI canister, compositions as described herein can inhibit or reduce one or more of the following: flocculation of active agent material; differential sedimentation or creaming of active agent particles and suspending particles; solution mediated transformation of active agent material; and loss of active agent to the surfaces of the container closure system, in particular the metering valve components. Such qualities work to achieve and preserve aerosol performance as the formulation is delivered from an MDI such that desirable fine particle fraction, fine particle dose, and delivered dose uniformity characteristics are achieved and substantially maintained throughout emptying of an MDI canister within which the formulation is contained. Additionally, compositions according to the present description can provide a stable formulation that provides consistent dosing characteristics, even for potent and highly potent active agents, while using a relatively simple HFC suspension medium that does not require modification by the addition of, for example, cosolvents, antisolvents, solubilizing agents, or adjuvants.

Providing a composition according to the present description may also simplify formulation, delivery, and dosing of the desired active agents. Without being bound by a particular theory, it is thought that by achieving a co-suspension of active agent particles and suspending particles, the delivery, physical stability, and dosing of an active agent contained within such a dispersion may be substantially controlled through control of the size, composition, morphology, and relative amount of the suspending particles, and is less dependent upon the size and morphology of the particles of active agent or on the properties of the propellant. Moreover, in specific embodiments, the pharmaceutical compositions described herein can be formulated with an HFC propellant or propellant system substantially free of antisolvents, solubilizing agents, cosolvents, or adjuvants.

In one embodiment, a composition described herein comprising a combination of two or more active agents may contain glycopyrronium bromide and formoterol fumarate as active agents. In one embodiment, a composition described herein comprising a combination of two or more active agents may contain budesonide, glycopyrronium bromide and formoterol fumarate as active agents. In one embodiment, a composition described herein comprising a combination of two or more active agents may contain albuterol sulfate and budesonide as active agents. In one embodiment, a composition described herein comprising a combination of two or more active agents may contain budesonide and formoterol fumarate as active agents. In one embodiment, a composition described herein comprising a combination of two or more active agents may contain budesonide, glycopyrronium bromide, formoterol fumarate and roflumilast as active agents.

In one embodiment, a composition described herein comprising a combination of two or more active agents may contain umeclidinium bromide, vilanterol trifenatate and fluticasone furoate as active agents. In another embodiment, a composition described herein comprising a combination of two or more active agents may contain umeclidinium bromide and vilanterol trifenatate as active agents. In one embodiment, a composition described herein comprising a combination of two or more active agents may contain glycopyrronium bromide, indacaterol acetate and mometasone furoate as active agents. In another embodiment, a composition described herein comprising a combination of two or more active agents may contain glycopyrronium bromide and indacaterol acetate as active agents. In one embodiment, a composition described herein comprising a combination of two or more active agents may contain glycopyrronium bromide, formoterol and beclometasone dipropionateas active agents. Compositions formulated according to the present teachings can inhibit degradation of active agent included therein.

In some embodiments, compositions formulated according to the present teachings inhibit physical and/or chemical degradation of the active agents included therein. For example, in specific embodiments, the compositions described herein may inhibit one or more of chemical degradation, flocculation, aggregation, and solution mediated transformation of the active agents included in the compositions. The chemical and suspension stability provided by the compositions described herein provides for enhanced robustness in simulated use testing (SUT) as compared to conventional preparations. Simulated use testing includes storage of an MDI canister for five weeks at 25° C. and 75% relative humidity (RH), with no weekly cleaning of the device, and dispensing of the composition from the MDI at 25° C. and 50% RH. Enhanced robustness can take the form of consistency of shot weight (i.e., the weight of the composition dispensed upon activation of the MDI), low levels of propellant leakage, and desirable delivered dose uniformity throughout emptying of an MDI canister (“DDU”), even where the active agents to be delivered are highly potent and delivered at very low doses. For example, in some embodiments the compositions described herein exhibit less than about 10%, less than about 9%, less than about 8%, less than about 7%, less than about 6%, or less than about 5% reduced shot weight when delivered by MDI in SUT. In further embodiments, the compositions described herein exhibit less than about 1.0%, less than about 0.5%, less than about 0.4%, less than about 0.3%, less than about 0.2%, or less than about 0.1% weight loss in the MDI per year at 20° C. and 60% RH. In still further embodiments, the compositions described herein exhibit a DDU of ±20% or better, ±15% or better, or ±10% or better, throughout emptying of the MDI canister. Moreover, compositions according to the present description exhibit enhance robustness by substantially preserving FPF and FPD performance throughout emptying of an MDI canister, even after being subjected to accelerated degradation conditions. For example, in some embodiments, the compositions described herein are dispensed from an MDI at a FPF that is maintained within about 85% or within about 95% of the initial FPF. Compositions described herein provide the added benefit of achieving such performance while being formulated using HFC propellants, e.g., HFC-152a. In specific embodiments, the compositions described herein achieve one or more of a targeted DDU, FPF, or FPD, while being formulated with suspension medium including only one or more HFC propellants and without the need to modify the characteristics of the propellant, such as by the addition of, for example, one or more cosolvent, antisolvent, solubilizing agent, adjuvant, or other propellant modifying material.

### Suspension Medium

The suspension medium included in a composition described herein includes one or more propellants. In general, suitable propellants for use as suspension mediums are those propellant gases that can be liquefied under pressure at room temperature, and upon inhalation or topical use, are safe and toxicologically innocuous. Additionally, it is desirable that the selected propellant be relatively non-reactive with the suspending particles or active agent particles. In the past, compositions for delivery by MDIs were typically formulated using chlorofluorocarbon (CFC) propellants, hydrofluoroalkanes (HFAs, e.g., HFA-134a and HFA-227ea), or perfluorinated compounds (PFCs). 1,1-Difluoroethane (HFC-152a) is considered more environmentally friendly, but several barriers existed to the use of HFC-152a in MDI formulations given the marked difference between HFC-152a and other propellants. Moreover, extensive experimentation would be needed to identify a formulation that would deliver the desired doses of active agent particles with desirable DDU and consistent FPF values.

As shown in Table A below, the physiochemical properties vary widely among different propellants.

TABLE A
Propellant Properties:

| | Propellant | | | | |
|---|---|---|---|---|---|
| | HFA-134a | HFA-227ea | HFC-152a | HFO-1234ze | HFO-1234yf |
| Chemical Formula | C₂H₂F₄ | C₃HF₇ | C₂H₄F₂ | C₃H₂F₄ | C₃H₂F₄ |
| Molecular Weight | 102 | 170 | 66 | 114 | 114 |
| (g/mol) | | | | | |
| Liquid Density @, | 1.23 | 1.41 | 0.91 | 1.18 | 1.11 |
| 20° C. (g/mL) | | | | | |
| Dipole Moment | 2.06 | 1.46 | 2.30 | 1.44 | 2.54 |
| (Debye) | | | | | |
| Surface Tension @ | 8.09 | 7.50 | 10.4 | 8.9 | 6.8 |
| 20° C. (mN/m) | | | | | |
| Boiling Point (° C.) | −26.1 | −16.5 | −24.7 | −19.0 | −29.5 |
| Liquid Viscosity @ | 0.211 | 0.267 | 0.171 | 3 | 0.164 |
| 20° C. (mPa · S) | | | | 0.206 | |
| Vapor Pressure @ | 570 | 390 | 510 | 427 | 592 |
| 20° C. (kPa) | | | | | |
| Water Solubility in | 1100@25 | 610@25 | 2100@25 | 225@20 | 200@24 |
| Propellant (ppm) | | | | | |
| Latent Heat @ | 177.7 | 110.97 | 279.1 | 166.8 | 145.4 |
| 25° C. (KJ/kg) | | | | | |
| Latent Heat @ | 216.7 | 131.4 | 318.4 | 195.43 | 180.5 |
| Boiling Point (kJ/kg) | | | | | |

Surprisingly, it was found that for compositions comprising active agent particles and suspending particles as described herein, MI formulations comprising HFC-152a propellant are suitable for use as inhalant medicine, despite the fact that HFC-152a and other propellants, e.g., HFAs, have significantly different structures and properties.

In some embodiments, the propellant is a pharmaceutical grade HFC-152a. The term “pharmaceutical grade propellant,” as used herein, indicates a propellant that is in compliance with the GMP regulations for use in humans. For example, the pharmaceutical grade propellant is consistent with the major health authorities' guidelines, such as FDA's or EMA's Guideline on The Pharmaceutical Quality of Inhalation and Nasal Products, and its specification as an excipient has been established to ensure the quality and safety of the propellant, e.g., HFC-152a, for pharmaceutical product use. The specification tests include propellant identity, appearance, assay, acidity, total residue, moisture content, related impurities, and unrelated impurities. Stability studies are also in progress to demonstrate long-term physicochemical stability. In some embodiments, the pharmaceutical grade HFC-152a has a purity of at least about 99.90%. In some embodiments, the propellant is pharmaceutical grade HFC-152a having a purity of about 99.90%, about 99.91%, about 99.92%, about 99.93%, about 99.94%, about 99.95%, or higher. Pharmaceutical grade HFC-152a is suitable for use as a propellant due to both its overall purity and the absence or low concentration of specific impurities. In some embodiments, the pharmaceutical grade HFC-152a contains about 10 ppm, about 9 ppm, about 8 ppm, about 7 ppm, about 6 ppm, about 5 ppm, or less of any one of the following impurities: HFO-1234yf, HFO-1234ze(Z), HFC-125, CFC-11, HFC-245cb, HFO-1225ye(Z) or HFO-1225ye(E), CFC-113, and CFC-114. In some embodiments, the pharmaceutical grade HFC-152a contains about 150 ppm, about 140 ppm, about 130 ppm, about 120 ppm, about 110 ppm, about 100 ppm, or less of HCFC-124.

In some embodiments, the suspension medium may be formed of a single propellant. In certain embodiments, certain vapor pressure compounds are present in a relatively low level. Such compounds may be associated with the suspending particles.

In some embodiments, the suspension medium may be formed of a propellant or propellant system that is substantially free of additional materials, including, for example, antisolvents, solubilizing agents, stabilizing agents, cosolvents, or adjuvants.

In some embodiments, the present pharmaceutical composition, which comprises a propellant of pharmaceutical grade HFC-152a; a plurality of active agent particles; and a plurality of phospholipid particles, exhibits similar or comparable bioavailability of the active agent(s) compared to a reference pharmaceutical composition, which comprises a propellant of pharmaceutical grade HFA-134a; a plurality of active agent particles; and a plurality of phospholipid particles. As used herein, a “reference pharmaceutical composition” means an alternative pharmaceutical composition which contains the same active agent particles and the same suspending particles as the present pharmaceutical composition except the propellant. For example, the present pharmaceutical composition and the reference pharmaceutical composition comprise the same active agent particles and the same phospholipids particles, but the reference pharmaceutical composition comprises a propellant of pharmaceutical grade HFA-134a, while the present pharmaceutical composition comprises a propellant of pharmaceutical grade HFC-152a. HFA-134a is a hydrofluoroalkane (HFA) with the chemical name: 1,1,1,2-tetrafluoroethane. HFA-134a has been used as a propellant in metered dose inhalers. As used herein, “bioavailability” means the proportion of an active agent which enters the circulation when introduced into the body through the lungs. In one embodiment, similar or comparable bioavailability can be shown, wherein the ratio of the geometric mean of logarithmic transformed Cmax, AUCinf or AUClast for the two products (e.g., the present pharmaceutical composition and the reference pharmaceutical composition) are about 0.80 to about 1.25 with or without the 90% confidence interval (CI) limits.

In some embodiments, the present pharmaceutical composition exhibits Cmax, AUCinf or AUClast of any one or more of the active agents, which is 80% to 125% of Cmax, AUCinf or AUClast of the one or more of the active agents of a reference pharmaceutical composition with geometric means ratios (GMR). In some embodiments, the present pharmaceutical composition comprises a propellant of pharmaceutical grade HFC-152a; a plurality of active agent particles; and a plurality of phospholipid particles comprising perforated microstructures, while the reference pharmaceutical composition comprises a propellant of pharmaceutical grade HFA-134a; a plurality of active agent particles; and a plurality of phospholipid particles comprising perforated microstructures. In some embodiments, the present pharmaceutical composition and the reference pharmaceutical composition are both administered by actuating metered dose inhalers, wherein each actuation of the present pharmaceutical composition provides the same delivered dose of the active agent(s) as each actuation of the reference pharmaceutical composition does. In some embodiments, the active agent particles comprise an active agent selected from a long-acting muscarinic antagonist (LAMA), a long-acting β2-agonists (LABA), a short-acting beta-agonists (SABA), an inhaled corticosteroid (ICS), and a non-corticosteroid anti-inflammatory agent as described herein.

As used herein, Cmax, AUCinf and AUClast are pharmacokinetic measures used to determine active agent dosing. As used herein, Cmax means the highest concentration of an active agent in the blood after a dose is administered, e.g., via inhalation. As used herein, the area under the curve (AUC) is the definite integral of a curve that describes the variation of an active agent concentration in blood plasma as a function of time. As used herein, AUCinf means area under the curve from the time of dosing to the last measurable concentration and extrapolated to infinity. As used herein, AUClast means area under the curve from the time of dosing to the last measurable concentration.

In some embodiments, the present pharmaceutical composition exhibits Cmax of budesonide, which is 80% to 125% of Cmax of budesonide of a reference pharmaceutical composition. In some embodiments, the present pharmaceutical composition exhibits Cmax of glycopyrrolate, which is 80% to 125% of Cmax of glycopyrrolate of a reference pharmaceutical composition. In some embodiments, the present pharmaceutical composition exhibits Cmax of formoterol, which is 80% to 125% of Cmax of formoterol of a reference pharmaceutical composition. In some embodiment, Cmax of budesonide is the geometric mean of logarithmic transformed value. In some embodiments, the present pharmaceutical composition exhibits Cmax of budesonide and formoterol, which is 80% to 125% of Cmax of budesonide and formoterol of a reference pharmaceutical composition. In some embodiments, the present pharmaceutical composition exhibits Cmax of budesonide and albuterol, which is 80% to 125% of Cmax of budesonide and albuterol of a reference pharmaceutical composition. In some embodiments, the present pharmaceutical composition exhibits Cmax of glycopyrrolate and formoterol, which is 80% to 125% of Cmax of glycopyrrolate and formoterol of a reference pharmaceutical composition. In some embodiments, the present pharmaceutical composition exhibits Cmax of budesonide, glycopyrrolate and formoterol, which is 80% to 125% of Cmax of budesonide, glycopyrrolate and formoterol of a reference pharmaceutical composition. In some embodiments, the present pharmaceutical composition exhibits Cmax of budesonide, glycopyrrolate, formoterol and roflumilast, which is 80% to 125% of Cmax of budesonide, glycopyrrolate, formoterol and roflumilast of a reference pharmaceutical composition.

In some embodiments, the present pharmaceutical composition exhibits AUCinf of budesonide, which is 80% to 125% of AUCinf of budesonide of a reference pharmaceutical composition. In some embodiments, the present pharmaceutical composition exhibits AUCinf of glycopyrrolate, which is 80% to 125% of AUCinf of glycopyrrolate of a reference pharmaceutical composition. In some embodiments, the present pharmaceutical composition exhibits AUCinf of formoterol, which is 80% to 125% of AUCinf of formoterol of a reference pharmaceutical composition. In some embodiment, AUCinf of budesonide is the geometric mean of logarithmic transformed value. In some embodiments, the present pharmaceutical composition exhibits AUCinf of budesonide and formoterol, which is 80% to 125% of AUCinf of budesonide and formoterol of a reference pharmaceutical composition. In some embodiments, the present pharmaceutical composition exhibits AUCinf of budesonide and albuterol, which is 80% to 125% of AUCinf of budesonide and albuterol of a reference pharmaceutical composition. In some embodiments, the present pharmaceutical composition exhibits AUCinf of glycopyrrolate and formoterol, which is 80% to 125% of AUCinf of glycopyrrolate and formoterol of a reference pharmaceutical composition. In some embodiments, the present pharmaceutical composition exhibits AUCinf of budesonide, glycopyrrolate and formoterol, which is 80% to 125% of AUCinf of budesonide, glycopyrrolate and formoterol of a reference pharmaceutical composition. In some embodiments, the present pharmaceutical composition exhibits AUCinf of budesonide, glycopyrrolate, formoterol and roflumilast, which is 80% to 125% of AUCinf of budesonide, glycopyrrolate, formoterol and roflumilast of a reference pharmaceutical composition.

In some embodiments, the present pharmaceutical composition exhibits AUClast of budesonide, which is 80% to 125% of AUClast of budesonide of a reference pharmaceutical composition. In some embodiments, the present pharmaceutical composition exhibits AUClast of glycopyrrolate, which is 80% to 125% of AUClast of glycopyrrolate of a reference pharmaceutical composition. In some embodiments, the present pharmaceutical composition exhibits AUClast of formoterol, which is 80% to 125% of AUClast of formoterol of a reference pharmaceutical composition. In some embodiment, AUClast of budesonide is the geometric mean of logarithmic transformed value. In some embodiments, the present pharmaceutical composition exhibits AUClast of budesonide and formoterol, which is 80% to 125% of AUClast of budesonide and formoterol of a reference pharmaceutical composition. In some embodiments, the present pharmaceutical composition exhibits AUClast of budesonide and albuterol, which is 80% to 125% of AUClast of budesonide and albuterol of a reference pharmaceutical composition. In some embodiments, the present pharmaceutical composition exhibits AUClast of glycopyrrolate and formoterol, which is 80% to 125% of AUClast of glycopyrrolate and formoterol of a reference pharmaceutical composition. In some embodiments, the present pharmaceutical composition exhibits AUClast of budesonide, glycopyrrolate and formoterol, which is 80% to 125% of AUClast of budesonide, glycopyrrolate and formoterol of a reference pharmaceutical composition. In some embodiments, the present pharmaceutical composition exhibits AUClast of budesonide, glycopyrrolate, formoterol and roflumilast, which is 80% to 125% of AUClast of budesonide, glycopyrrolate, formoterol and roflumilast of a reference pharmaceutical composition.

### Active Agent Particles

The active agent particles included in the compositions described herein are formed of a material capable of being dispersed and suspended within the suspension medium and are sized to facilitate delivery of respirable particles from the composition. In one embodiment, therefore, the active agent particles are provided as micronized particles wherein at least 90% of the active agent particles by volume exhibit an optical diameter of about 7 μm or less. In some embodiments, at least 90% of the active agent particles by volume exhibit an optical diameter of about 5 μm or less. In other embodiments, at least 90% of the active agent particles by volume exhibit an optical diameter selected from a range of about 7 μm to about 1 μm, about 5 μm to about 2 μm, and about 3 μm to about 2 μm. In further embodiments, at least 90% of the active agent particles by volume exhibit an optical diameter selected from 6 μm or less, 5 μm or less, 4 μm or less, or 3 μm or less. In another embodiment, the active agent particles are provided as micronized particles wherein at least 50% of the active agent particles by volume exhibit an optical diameter of about 4 μm or less. In further embodiments, the active agent particles are provided as micronized particles wherein at least 50% of the active agent particles by volume exhibit an optical diameter selected from about 3 μm or less, about 2 μm or less, about 1.5 μm or less, and about 1 μm or less. In still further embodiments, the active agent particles are provided as micronized particles wherein at least 50% of the active agent particles by volume exhibit an optical diameter selected from a range of about 4 μm to about 1 μm, about 3 μm to about 1 μm, about 2 μm to about 1 μm, about 1.3 μm, and about 1.9 μm.

In certain embodiments, the active agent particles comprise glycopyrrolate and at least 90% of the active agent particles by volume exhibit an optical diameter of about 7 μm or less. In certain embodiments, the active agent particles comprise budesonide and at least 90% of the active agent particles by volume exhibit an optical diameter of about 7 μm or less. In certain embodiments, the active agent particles comprise formoterol and at least 90% of the active agent particles by volume exhibit an optical diameter of about 5 μm or less. In certain embodiments, the active agent particles comprise albuterol and at least 90% of the active agent particles by volume exhibit an optical diameter of about 5 μm or less.

The active agent particles may be formed entirely of active agent or they may be formulated to include one or more active agents in combination with one or more excipients or adjuvants. In specific embodiments, an active agent present in the active agent particles may be entirely or substantially crystalline. In another embodiment, the active agent particles may include an active agent present in both crystal and amorphous states. In yet another embodiment, the active agent particles may include an active agent present in both crystal and amorphous states. In yet a further embodiment, where two or more active agents are present in active agent particles, at least one such active agent may be present in crystalline or substantially crystalline form and at least another active agent may be present in an amorphous state. In still another embodiment, where two or more active agents are present in active agent particles, each such active agent may be present in crystalline or substantially crystalline form. Where the active agent particles described herein include one or more active agents in combination with one or more excipients or adjuvants, the excipients and adjuvants can be selected based on the chemical and physical properties of the active agent used. Suitable excipients for formulation of active agent particles include, for example, lipid, phospholipids, carbohydrates, amino acids, organic salts, peptides, proteins, alditols, synthetic or natural polymers, or surfactant materials.

Any suitable process may be employed to achieve micronized active agent particles for inclusion in the compositions described herein. A variety of processes may be used to create active agent particles suitable for use in the formulations described herein, including, but not limited to, micronization by milling or grinding processes, crystallization or recrystallization processes, processes using precipitation from supercritical or near-supercritical solvents, spray drying, spray freeze drying, or lyophilization. Patent references teaching suitable methods for obtaining micronized active agent particles include, for example, U.S. Pat. Nos. 6,063,138, 5,858,410, 5,851,453, 5,833,891, 5,707,634, and International Patent Publication No. WO 2007/009164. Where the active agent particles include active agent material formulated with one or more excipient or adjuvant, micronized active agent particles can be formed using one or more of the preceding processes and such processes can be used to achieve active agent particles having a desired size distribution and particle configuration.

The active agent particles may be provided in any suitable concentration within the suspension medium. The active agent included in the active agent particles is substantially insoluble in the suspension medium. In some embodiments, the active agent, despite being substantially insoluble, exhibits measurable solubility in the suspension medium. However, even where the active agent exhibits measurable solubility in the suspension medium, the compositions described herein work to preserve the physical stability of such active agents. In particular, in specific embodiments, an active agent included in the compositions described herein may exhibit sufficient solubility in the suspension medium such that as much as 5% of the total active agent mass dissolves in the suspension medium. Alternatively, the solubility of an active agent may result in dissolution of as much as 1% of the total active agent mass in the suspension medium. In another embodiment, the solubility of an active agent may result in dissolution of as much as 0.5% of the total active agent mass in the suspension medium. In yet another embodiment, the solubility of an active agent may result in dissolution of as much as 0.05% of the total active agent mass in the suspension medium. In still another embodiment, the solubility of an active agent may result in dissolution of as much as 0.025% of the total active agent mass in the suspension medium.

A variety of therapeutic or prophylactic agents can be incorporated into the co-suspension compositions disclosed herein. Exemplary active agents include those that may be administered in the form of aerosolized medicaments, and active agents suitable for use in the compositions described herein include those that may be presented in a form or formulated in a manner which is dispersible within the selected suspension medium (e.g., is substantially insoluble or exhibits a solubility in the suspension medium that substantially maintains a co-suspension formulation), is capable of forming a co-suspension with the suspending particles, and is subject to respirable uptake in physiologically effective amounts. The active agents that may be utilized in forming the active agent particles described herein can have a variety of biological activities.

Examples of specific active agents that may be included in a composition according to the present description may for example, short-acting beta agonists (SABA), e.g., bitolterol, carbuterol, fenoterol, hexoprenaline, isoprenaline (isoproterenol), levosalbutamol, orciprenaline (metaproterenol), pirbuterol, procaterol, rimiterol, salbutamol (albuterol), terbutaline, tulobuterol, reproterol, ipratropium and epinephrine; long-acting β2 adrenergic receptor agonist (“LABA”), e.g., bambuterol, clenbuterol, formoterol, and salmeterol; ultra long-acting β2 adrenergic receptor agonists, e.g., carmoterol, milveterol, indacaterol, and saligenin- or indole-containing and adamantyl-derived β2 agonists; corticosteroids, e.g., beclomethasone, budesonide, ciclesonide, flunisolide, fluticasone, methyl-prednisolone, mometasone, prednisone and triamcinolone; anti-inflammatories, e.g. fluticasone propionate, beclomethasone dipropionate, flunisolide, budesonide, tripedane, cortisone, prednisone, prednisilone, dexamethasone, betamethasone, or triamcinolone acetonide; antitussives, e.g., noscapine; bronchodilators, e.g., ephedrine, adrenaline, fenoterol, formoterol, isoprenaline, metaproterenol, salbutamol, albuterol, salmeterol, terbutaline; and muscarinic antagonists, including long-acting muscarinic antagonists (“LAMA”), e.g., glycopyrrolate, dexpirronium, scopolamine, tropicamide, pirenzepine, dimenhydrinate, tiotropium, darotropium, aclidinium, trospium, ipatropium, atropine, benzatropin, or oxitropium.

Where appropriate, the active agents provided in the composition, including but not limited to those specifically described herein, may be used in the form of salts (e.g., alkali metal or amine salts or as acid addition salts) or as esters, solvates (hydrates), derivatives, or a free base thereof. Additionally, the active agents may be in any crystalline form or isomeric form or mixture of isomeric forms, for example, as pure enantiomers, a mixture of enantiomers, as racemates or as mixtures thereof. In this regard, the form of the active agents may be selected to optimize the activity and/or stability of the active agent and/or to minimize the solubility of the active agent in the suspension medium.

Because the compositions disclosed enable the reproducible delivery of very low doses of active agents, in certain embodiments, the active agent included in the compositions described herein may be selected from one or more potent or highly potent active agents. For example, in certain embodiments, the compositions described herein may include one or more potent active agents that are to be delivered at a dose selected from between about 100 μg and about 100 mg, about 100 μg and about 10 mg, and about 100 μg and 1 mg per actuation of an MDI. In other embodiments, the compositions described herein may include one or more potent or highly potent active agents that are to be delivered at a dose selected from up to about 80 μg, up to about 40 μg, up to about 20 μg, between about 10 μg and about 100 μg, between about 5 μg and about 50 μg, and between about 1 μg and about 10 μg per actuation of an MDI. Additionally, in certain embodiments, the compositions described herein may include one or more highly potent active agents that are to be delivered at a dose selected from between about 0.1 and about 2 μg, about 0.1 and about 1 μg, and about 0.1 and about 0.5 μg per actuation of an MDI.

A composition as described herein may, if desired, contain a combination of two or more active agents. For example, a combination of two or more species of active agent particles may be co-suspended with a single species of suspending particles. Alternatively, a composition may include two or more species of active agent particles co-suspended with two or more different species of suspending particles. Even further, a composition as described herein may include two or more active agents combined within a single species of active agent particle. For example, where the active agent particles are formulated using one or more excipients or adjuvants in addition to the active agent material, such active agent particles may include individual particles that include two or more different active agents.

In certain embodiments, the active agent included in the compositions described herein is a LAMA active agent. Where the compositions include a LAMA active agent, in particular embodiments, the LAMA active agent may be selected from, for example, glycopyrrolate, dexpirronium, tiotropium, trospium, aclidinium, umeclidinium, and darotropium, including any pharmaceutically acceptable salts, esters, isomers or solvates thereof. In some embodiments, a LAMA active agent is present at a concentration in the range of about 0.04 mg/mL to about 2.25 mg/mL.

Glycopyrrolate can be used to treat inflammatory or obstructive pulmonary diseases and disorders such as, for example, those described herein. As an anticholinergic, glycopyrrolate acts as a bronchodilator and provides an antisecretory effect, which is a benefit for use in the therapy of pulmonary diseases and disorders characterized by increased mucus secretions. Glycopyrrolate is a quaternary ammonium salt. Where appropriate, glycopyrrolate may be used in the form of salts (e.g. alkali metal or amine salts, or as acid addition salts) or as esters or as solvates (hydrates). Additionally, the glycopyrrolate may be in any crystalline form or isomeric form or mixture of isomeric forms, for example a pure enantiomer, a mixture of enantiomers, a racemate or a mixture thereof. In this regard, the form of glycopyrrolate may be selected to optimize the activity and/or stability of glycopyrrolate and/or to minimize the solubility of glycopyrrolate in the suspension medium. Suitable counter ions are pharmaceutically acceptable counter ions including, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenzoate, p-hydroxybenzoate, 1-hydroxynaphthalene-2-carboxylate, 3-hydroxynaphthalene-2-carboxylate, methanesulfonate and benzenesulfonate. In particular embodiments of the compositions described herein, the bromide salt of glycopyrrolate, namely 3-[(cyclopentyl-hydroxyphenylacetyl)oxy]-1,1-dimethylpyrrolidinium bromide, also referred to as (RS)-[3-(SR)-Hydroxy-1,1-dimethylpyrrolidinium bromide]α-cyclopentylmandelate, is used and can be prepared according to the procedures set out in U.S. Pat. No. 2,956,062.

Where the compositions described herein include glycopyrrolate, in certain embodiments, the compositions may include sufficient glycopyrrolate to provide a target delivered dose selected from between about 1 μg and about 200 μg per actuation of an MDI, about 5 μg and about 150 μg per actuation of an MDI, about 10 μg and 100 μg per actuation of an MDI, about 5 μg and about 50 μg per actuation of an MDI, between about 2 μg and about 25 μg per actuation of an MDI, and between about 6 μg and about 15 μg per actuation of an MDI. In other such embodiments, the formulations include sufficient glycopyrrolate to provide a dose selected from up to about 200 μg, up to about 150 μg, up to about 75 μg, up to about 40 μg, up to about 20 μg, or up to about 10 μg per actuation. In yet further embodiments, the formulations include sufficient glycopyrrolate to provide a dose selected from about 2 μg per actuation, about 5 μg per actuation, about 7 μg per actuation, about 9 μg per actuation, about 18 μg per actuation, 36 μg per actuation or about 72 μg per actuation. In order to achieve targeted delivered doses as described herein, where compositions described herein include glycopyrrolate as the active agent, in specific embodiments, the amount of glycopyrrolate included in the compositions may be selected from, for example, between about 0.04 mg/mL and about 2.25 mg/mL.

In other embodiments, tiotropium, including any pharmaceutically acceptable salts, esters, isomers or solvates thereof, may be selected as a LAMA active agent for inclusion in a composition as described herein. Tiotropium is a known, long-acting anticholinergic drug suitable for use in treating diseases or disorders associated with pulmonary inflammation or obstruction, such as those described herein. Tiotropium, including crystal and pharmaceutically acceptable salt forms of tiotropium, is described, for example, in U.S. Pat. Nos. 5,610,163, RE39,820, 6,777,423, and 6,908,928. Where the compositions described herein include tiotropium, in certain embodiments, the compositions may include sufficient tiotropium to provide a delivered dose selected from between about 2.5 μg and about 50 μg, about 4 μg and about g per actuation, and about 2.5 μg and about 20 μg, about 10 μg and about 20 μg, and about 2.5 μg and about 10 μg per actuation of an MDI. In other such embodiments, the formulations include sufficient tiotropium to provide a delivered dose selected from up to about 50 μg, up to about 20 μg, up to about 10 μg, up to about 5 μg, or up to about 2.5 μg per actuation of an MDI. In yet further embodiments, the formulations include sufficient tiotropium to provide a delivered dose selected from about 3 μg, 6 μg, 9 μg, 18 μg, and 36 μg per actuation of the MDI. In order to achieve delivered doses as described herein, where compositions described herein include tiotropium as the active agent, in specific embodiments, the amount of tiotropium included in the compositions may be selected from, for example, between about 0.01 mg/mL and about 0.5 mg/mL.

In certain embodiments, the compositions described herein include a LABA active agent. In such embodiments, a LABA active agent can be selected from, for example, bambuterol, clenbuterol, formoterol, salmeterol, carmoterol, milveterol, indacaterol, vilanterol, and saligenin- or indole-containing and adamantyl-derived β2 agonists, and any pharmaceutically acceptable salts, esters, isomers or solvates thereof. In some embodiments a LABA active agent is present at a concentration in the range of about 0.01 mg/mL to about 1 mg/mL.

In certain such embodiments, formoterol is selected as the LABA active agent. Formoterol can be used to treat inflammatory or obstructive pulmonary diseases and disorders such as, for example, those described herein. Formoterol has the chemical name (±)-2-hydroxy-5-[(1RS)-1-hydroxy-2-[[(1RS)-2-(4-methoxyphenyl)-1-methylethyl]-amino]ethyl]formanilide, and is commonly used in pharmaceutical compositions as the racemic fumarate dihydrate salt. Where appropriate, formoterol may be used in the form of salts (e.g. alkali metal or amine salts or as acid addition salts) or as esters or as solvates (hydrates). Additionally, the formoterol may be in any crystalline form or isomeric form or mixture of isomeric forms, for example a pure enantiomer, a mixture of enantiomers, a racemate or a mixture thereof. In this regard, the form of formoterol may be selected to optimize the activity and/or stability of formoterol and/or to minimize the solubility of formoterol in the suspension medium. Pharmaceutically acceptable salts of formoterol include, for example, salts of inorganic acids such as hydrochloric, hydrobromic, sulfuric and phosphoric acids, and organic acids such as fumaric, maleic, acetic, lactic, citric, tartaric, ascorbic, succinic, glutaric, gluconic, tricarballylic, oleic, benzoic, p-methoxybenzoic, salicylic, o- and p-hydroxybenzoic, p-chlorobenzoic, methanesulfonic, p-toluenesulfonic and 3-hydroxy-2-naphthalene carboxylic acids. Hydrates of formoterol are described, for example, in U.S. Pat. Nos. 3,994,974 and 5,684,199. Specific crystalline forms of formoterol and other β2 adrenergic receptor agonists are described, for example, in WO95/05805, and specific isomers of formoterol are described in U.S. Pat. No. 6,040,344.

In specific embodiments, the formoterol material utilized to form the formoterol particles is formoterol fumarate, and in one such embodiment, the formoterol fumarate is present in the dihydrate form. Formoterol fumarate may be referred to by the chemical name N-[2-Hydroxy-5-[(1RS)-1-hydroxy-2-[[(1RS)-2-(4-methoxyphenyl)-1-methylethyl]-amino]ethyl]phenyl]formamide (E)-2-butenedioate dehydrate. Where the compositions described herein include formoterol, in certain embodiments, the compositions described herein may include formoterol at a concentration that achieves a targeted delivered dose selected from between about 1 μg and about 30 μg, about 0.5 μg and about 10 μg, about 1 μg and about 10 μg, about 2 μg and 5 μg, about 2 μg and about 10 μg, about 3 μg and about 10 μg, about 5 μg and about 10 μg, and 3 μg and about 30 μg per actuation of an MDI. In other embodiments, the compositions described herein may include formoterol in an amount sufficient to provide a targeted delivered dose selected from up to about 30 μg, up to about 10 μg, up to about 5 μg, up to about 2.5 μg, up to about 2 μg, or up to about 1.5 μg per actuation. In yet further embodiments, the formulations include sufficient formoterol to provide a dose selected from about 2 μg per actuation, about 4.5 μg per actuation, about 4.8 μg per actuation, about 5 μg per actuation, about 10 μg per actuation, about 20 μg per actuation, or about 30 μg per actuation. In order to achieve targeted delivered doses as described herein, where compositions described herein include formoterol as the active agent, in specific embodiments, the amount of formoterol included in the compositions may be selected from, for example, between about 0.01 mg/mL and about 1 mg/mL, between about 0.01 mg/mL and about 0.5 mg/mL, and between about 0.03 mg/mL and about 0.4 mg/mL.

Where the pharmaceutical compositions described herein include a LABA active agent, in certain embodiments, the active agent may be salmeterol, including any pharmaceutically acceptable salts, esters, isomers or solvates thereof. Salmeterol can be used to treat inflammatory or obstructive pulmonary diseases and disorders such as, for example, those described herein. Salmeterol, pharmaceutically acceptable salts of salmeterol, and methods for producing the same are described, for example, in U.S. Pat. Nos. 4,992,474, 5,126,375, and 5,225,445.

Where salmeterol is included as a LABA active agent, in certain embodiments, the compositions described herein may include salmeterol at a concentration that achieves a delivered dose selected from between about 2 μg and about 120 μg, about 4 μg and about 40 μg, about 8 μg and 20 μg, about 8 μg and about 40 μg, about 20 μg and about 40 μg, and about 12 μg and about 120 μg per actuation of an MDI. In other embodiments, the compositions described herein may include salmeterol in an amount sufficient to provide a delivered dose selected from up to about 120 μg, up to about 40 μg, up to about 20 μg, up to about 10 μg, up to about 8 μg, or up to about 6 μg per actuation of an MDI. In order to achieve targeted delivered doses as described herein, where compositions described herein include salmeterol as the active agent, in specific embodiments, the amount of salmeterol included in the compositions may be selected from, for example, between about 0.04 mg/mL and about 4 mg/mL, between about 0.04 mg/mL and about 2.0 mg/mL, and between about 0.12 mg/mL and about 0.8 mg/mL.

Where the pharmaceutical compositions described herein include a SABA active agent, in certain embodiments, the active agent may be bitolterol, carbuterol, fenoterol, hexoprenaline, isoprenaline (isoproterenol), levosalbutamol, orciprenaline (metaproterenol), pirbuterol, procaterol, rimiterol, albuterol (salbutamol), terbutaline, tulobuterol, reproterol, and epinephrine, including any pharmaceutically acceptable salts, esters, isomers, or solvates thereof. In certain such embodiments, albuterol is selected as the SABA active agent. Albuterol has the chemical name α¹-[(tert-butylamino)methyl]4-hydroxy-m-xylene-α,α′-diol and has an empirical formula of C₁₃H₂₁NO₃. Albuterol can be used to treat inflammatory or obstructive pulmonary diseases and disorders such as, for example, those described herein. Albuterol, pharmaceutically acceptable salts of albuterol (such as albuterol sulfate), and methods for producing the same are described, for example, in U.S. Pat. No. 3,705,233.

Where albuterol is included as a SABA active agent, in certain embodiments, the compositions described herein may include albuterol at a concentration that achieves a delivered dose selected from between about 10 μg and about 200 μg, about g and about 300 μg, about 30 μg and 150 μg, about 50 μg and about 200 μg, about 30 μg and about 100 μg, and about 1 μg and about 300 μg per actuation of an MDI. In other embodiments, the compositions described herein may include albuterol in an amount sufficient to provide a delivered dose selected from up to about 300 μg, up to about 200 μg, up to about 150 μg, up to about 100 μg, up to about 50 μg, up to about 30 μg, up to about 20 μg, or up to about 10 μg per actuation of an MDI. In yet further embodiments, the formulations include sufficient albuterol to provide a dose selected from about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, or about 150 μg per actuation. In order to achieve targeted delivered doses as described herein, where compositions described herein include albuterol as the active agent, in specific embodiments, the amount of albuterol included in the compositions may be selected from, for example, between about 0.1 mg/mL and about 10 mg/mL, between about 0.1 mg/mL and about 5 mg/mL, and between about 0.3 mg/mL and about 4 mg/mL.

In still other embodiments, the compositions described herein include a corticosteroid, such as an inhaled corticosteroid (ICS). Such active agents may be selected from, for example, beclomethasone, budesonide, ciclesonide, flunisolide, fluticasone, methylprednisolone, mometasone, prednisone and triamcinolone, and any pharmaceutically acceptable salts, esters, isomers or solvates thereof. In some embodiments, an ICS active agent is present at a concentration in the range of about 0.1 mg/mL to about 10 mg/mL.

Where the compositions include an ICS active agent, in particular embodiments, mometasone may be selected. Mometasone, pharmaceutically acceptable salts of mometasone, such as mometasone furoate, and preparation of such materials are known and described, for example, in U.S. Pat. Nos. 4,472,393, 5,886,200, and 6,177,560. Mometasone is suitable for use in treating diseases or disorders associated with pulmonary inflammation or obstruction, such as those described herein (see, e.g., U.S. Pat. Nos. 5,889,015, 6,057,307, 6,057,581, 6,677,322, 6,677,323 and 6,365,581).

Where the compositions described herein include mometasone, in particular embodiments, the compositions include mometasone, including any pharmaceutically acceptable salts, esters, isomers or solvates thereof, in an amount sufficient to provide a target delivered dose selected from between about 20 μg and about 400 μg, about 20 μg and about 200 μg, about 50 μg and about 200 μg, about 100 μg and about 200 μg, about 20 μg and about 100 μg, and about 50 μg and about 100 μg per actuation of an MDI. In still other embodiments, the compositions described herein may include mometasone, including any pharmaceutically acceptable salts, esters, isomers or solvates thereof, in an amount sufficient to provide a targeted delivered dose selected from up to about 400 μg, up to about 200 μg, or up to about 100 μg per actuation of an MDI.

In other embodiments, the compositions described herein include a corticosteroid selected from fluticasone and budesonide. Both fluticasone and budesonide are suitable for use in treatment of conditions associated with pulmonary inflammation or obstruction, such as those described herein. Fluticasone, pharmaceutically acceptable salts of fluticasone, such as fluticasone propionate, and preparation of such materials are known, and described, for example, in U.S. Pat. Nos. 4,335,121, 4,187,301, and U.S. Pat. Pub. No. US2008125407. Budesonide, which has the chemical name (RS)-11β,16α, 17,21-Tetrahydroxypregna-1,4-diene-3,20-dione cyclic 16,17-acetal with butyraldehyde, is also well known and described, for example, in U.S. Pat. No. 3,929,768. In certain embodiments, compositions described herein may include fluticasone, including any pharmaceutically acceptable salts, esters, isomers or solvates thereof, in an amount sufficient to provide a target delivered dose selected from between about 20 μg and about 200 μg, about 50 μg and about 175 μg, and between about 80 μg and about 160 μg per actuation of an MDI. In other embodiments, the compositions described herein may include fluticasone, including any pharmaceutically acceptable salts, esters, isomers or solvates thereof, in an amount sufficient to provide a targeted delivered dose selected from up to about 175 μg, up to about 160 μg, up to about 100 μg, or up to about 80 μg per actuation of an MDI. Where the compositions described herein include budesonide, in certain embodiments, the compositions described herein may include budesonide, including any pharmaceutically acceptable salts, esters, isomers or solvates thereof, at a concentration that achieves a targeted delivered dose selected from between about 30 μg and about 240 μg, about 30 μg and about 120 μg, between about 30 μg and about 100 μg, between about 50 μg and about 400 μg, between about 20 μg and about 600 μg, between about 50 μg and about 200 μg, between about 150 μg and about 350 μg, and between about 30 μg and about 50 μg per actuation of an MDI. In other embodiments, the compositions described herein may include budesonide, including any pharmaceutically acceptable salts, esters, isomers or solvates thereof, in an amount sufficient to provide a targeted delivered dose selected from up to about 240 μg, up to about 160 μg, up to about 120 μg, up to about 80 μg, or up to about 50 μg per actuation of an MDI. In yet further embodiments, the formulations include sufficient budesonide to provide a dose selected from about 20 μg per actuation, about 40 μg per actuation, about 80 μg per actuation, about 100 μg per actuation, about 160 μg per actuation, about 200 μg per actuation, or about 300 μg per actuation. In order to achieve targeted delivered doses as described herein, where compositions described herein include budesonide as the active agent, in specific embodiments, the amount of budesonide included in the compositions may be selected from, for example, between about 0.1 mg/mL and about 20 mg/mL, between about 0.1 mg/mL and about 5 mg/mL, and between about 0.3 mg/mL and about 6 mg/mL.

In yet further embodiments, the compositions described herein include a non-corticosteroid anti-inflammatory agent, such as a phosphodiesterase-4 (PDE-4) inhibitor and a Janus kinase (JAK) inhibitor. Such anti-inflammatory agents may be selected from, for example, roflumilast, apremilast, crisaborole, ruxolitinib, tofacitinib, oclacitinib, baricitinib, peficitinib, fedratinib, and upadacitinib or any pharmaceutically acceptable salts, esters, isomers or solvates thereof. Roflumilast, pharmaceutically acceptable salts of roflumilast, and preparation of such materials are known and described, for example, in U.S. Pat. Nos. 8,604,064, 9,145,365, and 9,321,726. Roflumilast is suitable for use in treating diseases or disorders associated with pulmonary inflammation or obstruction, such as those described herein. Roflumilast is sometimes used for the treatment of COPD, particularly severe COPD, and is available as an oral medication. Gastrointestinal side effects are common with oral administration of roflumilast.

Where the compositions described herein include roflumilast, in certain embodiments, the compositions described herein may include roflumilast, including any pharmaceutically acceptable salts, esters, isomers or solvates thereof, at a concentration that achieves a targeted delivered dose selected from between about 30 μg and about 240 μg, about 30 μg and about 120 μg, between about 30 μg and about 100 μg, between about 50 μg and about 400 μg, between about 20 μg and about 600 μg, between about 50 μg and about 200 μg, between about 150 μg and about 350 μg, and between about 30 μg and about 50 μg per actuation of an MDI. In other embodiments, the compositions described herein may include roflumilast, including any pharmaceutically acceptable salts, esters, isomers or solvates thereof, in an amount sufficient to provide a targeted delivered dose selected from up to about 240 μg, up to about 160 μg, up to about 120 μg, up to about 80 μg, or up to about 50 μg per actuation of an MDI. In yet further embodiments, the formulations include sufficient roflumilast to provide a dose selected from about 20 μg per actuation, about 40 μg per actuation, about 80 μg per actuation, about 100 μg per actuation, about 160 μg per actuation, about 200 μg per actuation, or about 300 μg per actuation. In order to achieve targeted delivered doses as described herein, where compositions described herein include roflumilast as the active agent, in specific embodiments, the amount of roflumilast included in the compositions may be selected from, for example, between about 0.1 mg/mL and about 20 mg/mL, between about 0.1 mg/mL and about 5 mg/mL, and between about 0.3 mg/mL and about 6 mg/mL.

The compositions described herein can be formulated to include (and deliver) a single active agent. Alternatively, the compositions described herein may include two or more active agents. In particular embodiments, where two or more active agents are included, the compositions described herein may include a combination of active agents selected from a combination of a LAMA and LABA active agents, a combination of LAMA and corticosteroid active agents, a combination of LAMA and SABA active agents, a combination of LAMA and non-corticosteroid anti-inflammatory active agents, a combination of LABA and SABA active agents, a combination of LABA and non-corticosteroid anti-inflammatory active agents, a combination of SABA and corticosteroid active agents, a combination of SABA and non-corticosteroid anti-inflammatory active agents, and a combination of LABA and corticosteroid active agents. In other embodiments, the compositions described herein may include three or more active agents. In certain such embodiments, the composition includes a combination of active agents selected from a combination of a LAMA, LABA, corticosteroid, and non-corticosteroid anti-inflammatory active agents active agents. For example, a composition as described herein may include a combination of active agents selected from a combination of glycopyrrolate and formoterol, a combination of formoterol and budesonide, a combination of budesonide and albuterol, a combination of glycopyrrolate, formoterol, and budesonide and a combination of glycopyrrolate, formoterol, budesonide, and roflumilast.

With the aid of the present disclosure, it will be appreciated by those having skill in the art that a wide variety of active agents may be incorporated into the suspensions disclosed herein. The above list of active agents is by way of example and not limitation.

### Suspending Particles

The suspending particles included in the compositions described herein work to facilitate stabilization and delivery of the active agent included in the compositions. Though various forms of suspending particles may be used, the suspending particles are typically formed from pharmacologically inert material that is acceptable for inhalation and is substantially insoluble in the propellant selected. Generally, the majority of suspending particles are sized within a respirable range. In particular embodiments, therefore, the MMAD of the suspending particles will not exceed about 10 μm but is not lower than about 500 nm. In an alternative embodiment, the MMAD of the suspending particles is between about 5 μm and about 750 nm. In yet another embodiment, the MMAD of the suspending particles is between about 1 μm and about 3 μm. When used in an embodiment for nasal delivery from an MDI, the MMAD of the suspending particles is between 10 μm and 50 μm.

In order to achieve respirable suspending particles within the MMAD ranges described, the suspending particles will typically exhibit a volume median optical diameter between about 0.2 μm and about 50 μm. In one embodiment, the suspending particles exhibit a volume median optical diameter that does not exceed about 25 μm. In another embodiment, the suspending particles exhibit a volume median optical diameter selected from between about 0.5 μm and about 15 μm, between about 1.5 μm and about 10 μm, and between about 2 μm and about 5 μm.

The concentration of suspending particles included in a composition according to the present description can be adjusted, depending on, for example, the amount of active agent particles and suspension medium used. In one embodiment, the suspending particles are included in the suspension medium at a concentration selected from about 0.1 mg/mL to about 15 mg/mL, about 0.1 mg/mL to about 10 mg/mL, 1 mg/mL to about 15 mg/mL, about 3 mg/mL to about 10 mg/mL, 5 mg/mL to about 8 mg/mL, and about 6 mg/mL. In another embodiment, the suspending particles are included in the suspension medium at a concentration of up to about 30 mg/mL. In yet another embodiment, the suspending particles are included in the suspension medium at a concentration of up to about 25 mg/mL.

The relative amount of suspending particles to active agent particles is selected to achieve a co-suspension as contemplated herein. A co-suspension composition may be achieved where the amount of suspending particles, as measured by mass, exceeds that of the active agent particles. For example, in specific embodiments, the ratio of the total mass of the suspending particles to the total mass of active agent particles may be between about 3:1 and about 15:1, or alternatively from about 2:1 and 8:1. Alternatively, the ratio of the total mass of the suspending particles to the total mass of active agent particles may be above about 1, such as up to about 1.5, up to about 5, up to about 10, up to about 15, up to about 17, up to about 20, up to about 30, up to about 40, up to about 50, up to about 60, up to about 75, up to about 100, up to about 150, and up to about 200, depending on the nature of the suspending particles and active agent particles used. In further embodiments, the ratio of the total mass of the suspending particles to the total mass of the active agent particles may be selected from between about 10 and about 200, between about 60 and about 200, between about 15 and about 60, between about 15 and about 170, between about 15 and about 60, about 16, about 60, and about 170.

In other embodiments, the amount of suspending particles, as measured by mass, is less than that of the active agent particles. For example, in particular embodiments, the mass of the suspending particles may be as low as 20% of the total mass of the active agent particles. However, in some embodiments, the total mass of the suspending particles may also approximate or equal the total mass of the active agent particles.

Suspending particles suitable for use in the compositions described herein may be formed of one or more pharmaceutically acceptable materials or excipients that are suitable for inhaled delivery and do not substantially degrade or dissolve in the suspension medium. In one embodiment, perforated microstructures, as defined herein, may be used as the suspending particles. Suspending particles and perforated microstructures for use as suspending particles, and methods for preparation thereof, are described in U.S. Pat. Nos. 8,815,258 and 9,463,161, and in U.S. Patent Application Publication 2011/0135737.

Phospholipids from both natural and synthetic sources may be used in preparing suspending particles comprising perforated microstructures suitable for use in the compositions described herein. In particular embodiments, the phospholipid chosen will have a gel to liquid crystal phase transition of greater than about 400° C. Exemplary phospholipids are relatively long chain (i.e., C16-C22) saturated lipids and may comprise saturated phospholipids, such as saturated phosphatidylcholines having acyl chain lengths of 16 C or 18 C (palmitoyl and stearoyl). Exemplary phospholipids include phosphoglycerides such as dipalmitoylphosphatidylcholine, disteroylphosphatidylcholine, diarachidoylphosphatidylcholine, dibehenoylphosphatidylcholine, diphosphatidyl glycerol, short-chain phosphatidylcholines, long-chain saturated phosphatidylethanolamines, long-chain saturated phosphatidylserines, long-chain saturated phosphatidylglycerols, and long-chain saturated phosphatidylinositols. Additional excipients are disclosed in International Patent Publication No. WO 96/32149 and U.S. Pat. Nos. 6,358,530, 6,372,258 and 6,518,239. In certain embodiments, the suspending particles are phospholipid particles comprising 1,2-Distearoyl-sn-glycero-3-phosphocholine (DSPC).

In another aspect, the suspending particles utilized in the compositions described herein may be selected to increase storage stability of the selected active agent, similar to that disclosed in International Patent Publication No. WO 2005/000267. For example, in one embodiment, the suspending particles my include pharmaceutically acceptable glass stabilization excipients having a Tg of at least 55° C., at least 75° C., or at least 100° C. Glass formers suitable for use in compositions described herein include, but are not limited to, one or more of thleucine, sodium citrate, sodium phosphate, ascorbic acid, inulin, cyclodextrin, polyvinyl pyrrolidone, mannitol, sucrose, trehalose, lactose, and, proline. Examples of additional glass-forming excipients are disclosed in U.S. Pat. Nos. RE 37,872, 5,928,469, 6,258,341, and 6,309,671. In particular embodiments, suspending particles may include a calcium salt, such as calcium chloride, as described, for example, in U.S. Pat. No. 7,442,388.

In certain embodiments, the suspending particles are perforated microstructures comprising DSPC and calcium chloride.

The suspending particles may be designed, sized and shaped as desired to provide desirable stability and active agent delivery characteristics. In one exemplary embodiment, the suspending particles comprise perforated microstructures as described herein. Where perforated microstructures are used as suspending particles in the compositions described herein, they may include at least one of the following: lipids, phospholipids, nonionic detergents, nonionic block copolymers, ionic surfactants, biocompatible fluoronated surfactants and combinations thereof, particularly those approved for pulmonary use. Specific surfactants that may be used in the preparation of perforated microstructures include poloxamer 188, poloxamer 407 and poloxamer 338. Other specific surfactants include oleic acid or its alkali salts. In one embodiment, the perforated microstructures include greater than about 10% w/w surfactant.

Furthermore, suspending particles as described herein may include bulking agents, such as polymeric particles. Polymeric polymers may be formed from biocompatible and/or biodegradable polymers, copolymers or blends. In one embodiment, polymers capable of forming aerodynamically light particles may be used, such as functionalized polyester graft copolymers and biodegradable polyanhydrides. For example, bulk eroding polymers based on polyesters including poly(hydroxy acids) can be used. Polyglycolic acid (PGA), polylactic acid (PLA) or copolymers thereof may be used to form suspending particles. The polyester may include a charged or functionalizable group, such as an amino acid. For example, suspending particles may be formed of poly(D,ι-lactic acid) and/or poly(D,ι-lactic-co-glycolic acid) (PLGA), which incorporate a surfactant such as DPPC.

Other potential polymer candidates for use in suspending particles may include polyamides, polycarbonates, polyalkylenes such as polyethylene, polypropylene, poly(ethylene glycol), poly(ethylene oxide), poly(ethylene terephthalate), polyvinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

In an embodiment of a composition as described herein that includes one or more of glycopyrrolate, formoterol, budesonide, and albuterol as an active agent, the ratio of the total mass of the suspending particles to the total mass of the active agent particles may be selected from between about 1 and about 20, between about 1 and about 15, between about 1.5 and about 10, between about 2.5 and about 15, between about 2.5 and about 10, between about 2.5 and about 8, between about 10 and about 30, between about 15 and about 25, between about 10 and about 200, between about 50 and about 125, and between about 5 and about 50.

In some embodiments, suspending particles may be prepared by forming an oil-in-water emulsion, using a fluorocarbon oil (e.g., perfluorooctyl bromide, perfluorodecalin) which may be emulsified using a surfactant such as a long chain saturated phospholipid. The resulting perfluorocarbon in water emulsion may be then processed using a high pressure homogenizer to reduce the oil droplet size. The perfluorocarbon emulsion may be fed into a spray dryer. As is well known, spray drying is a one-step process that converts a liquid feed to a dried particulate form. Spray drying has been used to provide powdered pharmaceutical material for various administrative routes, including inhalation. In the context of spray drying, a fluorocarbon oil such as described above may function as a blowing agent. Operating conditions of the spray dryer (such as inlet and outlet temperature, feed rate, atomization pressure, flow rate of the drying air and nozzle configuration) can be adjusted to produce the desired particle size producing a yield of the resulting dry microstructures. Such methods of producing exemplary perforated microstructures are disclosed in U.S. Pat. Nos. 8,815,258, 9,463,161, and U.S. Patent Application Publication 2011/0135737,

The compositions described herein may include two or more species of suspending particles. For example, the compositions described herein may include a single species of active agent particle and two or more species of suspending particles. Alternatively, in other embodiments, the compositions described herein may include two or more species of active agent particles combined with two or more species of suspending particles.

Compositions formulated according to the present teachings can inhibit degradation of active agent included therein. For example, in specific embodiments, the compositions described herein inhibit one or more of flocculation, aggregation and the solution mediated transformation of active agent material included in the compositions. The pharmaceutical compositions described herein are suited for respiratory delivery via and MDI in a manner that achieves desirable delivered dose uniformity (“DDU”) of each active agent included in a combination of two or more active agents, even with combinations including potent and highly potent actives. As is illustrated in detail in the Examples included herein, even when delivering very low doses of two or more active agents, compositions described herein can achieve a DDU of ±30%, or better, for each active agent throughout emptying of an MDI canister. In one such embodiment, compositions described herein achieve a DDU of ±25%, or better, for each active agent throughout emptying of an MDI canister. In another such embodiment, compositions described herein achieve a DDU for the active agent of ±20%, or better, for each active agent throughout emptying of an MDI canister. In further embodiments, compositions described herein achieve a DDU for the active agent of ±15%, or better, for each active agent throughout emptying of an MDI canister. In still further embodiments, compositions described herein achieve a DDU for the active agent of ±10%, or better, for each active agent throughout emptying of an MDI canister.

Pharmaceutical compositions described herein also serve to substantially preserve FPF and FPD performance throughout emptying of an MDI canister, even after being subjected to accelerated degradation conditions. For instance, compositions according to the present description maintain as much as 80%, 85%, 90%, 95%, or more, of the original FPF and FPD performance throughout emptying of an MDI canister, even after being subjected to accelerated degradation conditions. Compositions described herein provide the added benefit of achieving such performance while being formulated using non-CFC and non-HFA propellants and eliminating or substantially avoiding combination effects often experienced with compositions incorporating multiple active agents. In specific embodiments, the compositions described herein achieve one or all of a targeted DDU, FPF and FPD performance while being formulated with suspension medium including only one or more HFC propellant and without the need to modify the characteristics of the HFC propellant, such as by the addition of, for example, one or more cosolvent, antisolvent, solubilizing agent, adjuvant or other propellant modifying material.

### Methods

Compositions formulated according to the present teachings can inhibit degradation of the active agent included therein. For example, in specific embodiments, the compositions described herein inhibit one or more of flocculation, aggregation and Ostwald ripening of the active agent(s) included in the compositions. The stability provided by the compositions described herein allows the compositions to be dispensed in a manner that achieves desirable delivered dose uniformity throughout emptying of an MDI canister (“DDU”), even where the active agent to be delivered is highly potent and the delivered dose of the active agent is selected from, for example, less than one of 100 μg, 80 μg, 40 μg, 20 μg, 10 μg, 9 μg, 8 μg, 7 μg, 6 μg, 5 μg, 4 μg, 3 μg, 2 μg, 1 μg, 0.5 μg, and 0.1 μg per actuation of the MDI. As is described in detail in the Examples included herein, even at low doses of highly potent active agents, compositions described herein can achieve a DDU of ±30%, or better, for each of the active agents included in the composition. In an alternative embodiment, compositions described herein achieve a DDU of ±25%, or better, for each of the active agents included in the composition. In yet further embodiments, compositions described herein achieve a DDU of ±20%, or better, ±15%, or better, or ±10%, or better, for each of the active agents included in the composition.

Moreover, compositions according to the present description serve to substantially preserve FPF and FPD performance throughout emptying of an MDI canister, even after being subjected to accelerated degradation conditions. For instance, compositions according to the present description maintain as much as 80%, 85%, 90%, 95%, or more, of the original FPF and FPD performance, even when they incorporate multiple active agents. Compositions described herein provide the added benefit of achieving such performance while being formulated using non-CFC and non-HFA propellants. In specific embodiments, the compositions described herein achieve desired one or all of a targeted DDU, FPF and FPD performance while being formulated with suspension medium including only one or more HFC propellant and without the need to modify the characteristics of the HFC propellant, such as by the addition of, for example, one or more cosolvent, antisolvent, solubilizing agent, adjuvant or other propellant modifying material.

The stability and physical characteristics of the compositions described herein support several methods. For example, in one embodiment, a method of formulating a pharmaceutical composition for respiratory delivery of an active agent is provided herein. The method involves the steps of providing a suspension medium comprising an HFC propellant, one or more species of active agent particles and one or more species of suspending particles, as described herein, and combining such constituents to form a composition wherein active agent particles associate with the suspending particles such that a co-suspension as described herein is formed. In one such embodiment, the association of the active agent particles and the suspending particles is such that they do not separate due to their different buoyancies in a propellant. As will be appreciated, a method of formulating a pharmaceutical composition as described herein can include providing two or more species of active agent particles in combination with one or more species of suspending particles. Alternatively, the method may include providing two or more suspending particles in combination with one or more species of active agent particles.

In further embodiments the compositions described herein support, for example, methods for forming stabilized formulations of active agents for pulmonary delivery, methods for preserving the FPF and/or FPD throughout emptying of an MDI canister, methods for pulmonary delivery of potent or highly potent active agents, and methods of achieving a DDU selected from ±30%, or better, ±25%, or better, ±20%, or better, ±15%, or better, and ±10%, or better, for potent and highly potent drugs administered through pulmonary delivery.

In methods involving pulmonary delivery of active agents using compositions described herein, the compositions may be delivered by an MDI. Therefore, in particular embodiments of such methods, an MDI loaded with a composition described herein is obtained, and the desired active agent is administered to a patient through pulmonary delivery through actuation of the MDI. For example, in one embodiment, after shaking the MDI device, the mouthpiece is inserted into a patient's mouth between the lips and teeth. The patient typically exhales deeply to empty the lungs and then takes a slow deep breath while actuating the cartridge of the MDI. When actuated, the specified volume of formulation travels to the expansion chamber, out the actuator nozzle and into a high-velocity spray that is drawn into the lungs of a patient. In some embodiments the dose of active agent delivered throughout emptying of an MDI canister is not more than 20% greater than the mean delivered dose and is not less than 20% less than the mean delivered dose. In some embodiments, the dose of active agent delivered throughout emptying of an MDI canister is not more than 15% greater or less than the mean delivered dose. In some embodiments, the dose of active agent delivered throughout emptying of an MDI canister is not more than 10% greater or less than the mean delivered dose.

In specific embodiments of methods for providing a stabilized formulation of active agent for pulmonary delivery, the present disclosure provides methods for inhibiting solution mediated transformation of an active agent in a pharmaceutical formulation for pulmonary delivery. In one embodiment, a suspension medium as described herein, such as a suspension medium formed by an HFC propellant, is obtained. Suspending particles are also obtained or prepared as described herein. One or more species of active agent particles as described herein are also obtained, and the suspension medium, suspending particles and active agent particles are combined to form a co-suspension wherein the active agent particles associate with suspending particles within the continuous phase formed by the suspension medium. When compared to the active agent contained in the same suspension medium in the absence of suspending particles, co-suspensions according to the present description have been found to exhibit a higher tolerance to solution mediated transformation and irreversible crystal aggregation, and thus can lead to improved stability and dosing uniformity, allowing the formulation of active agents that are somewhat physically unstable in the suspension medium alone.

In specific embodiments of methods for preserving the FPF and/or FPD provided by a pharmaceutical formulation for pulmonary delivery of a respirable co-suspension as described herein is provided which is capable of maintaining the FPD and/or the FPF to within ±20%,±15%, ±10%, or even ±5% the initial FPD and/or FPF, respectively, throughout emptying of an MDI canister. Such performance can be achieved even after the co-suspension is subjected to accelerated degradation conditions. In one embodiment, a suspension medium as described herein, such as a suspension medium formed by an HFC propellant, is obtained. Suspending particles are also obtained or prepared as described herein. One or more species of active agent particles as described herein are also obtained, and the suspension medium, suspending particles and active agent particles are combined to form a co-suspension wherein the active agent particles associate with suspending particles within the suspension medium. Even after exposure of such composition to one or more temperature cycling events, the co-suspension maintains an FPD or FPF within ±20%, ±15%, ±10%, or even ±5% of the respective values measured prior to exposure of the composition to the one or more temperature cycling events.

Methods for treating patients suffering from an inflammatory or obstructive pulmonary disease or condition are provided herein. In specific embodiments, such methods include pulmonary delivery of a therapeutically effective amount of a pharmaceutical composition described herein, and in certain such embodiments, pulmonary administration of the pharmaceutical composition is accomplished by delivering the composition using an MDI. In certain embodiments, the compositions, methods and systems described herein can be used to treat patients suffering from a disease or disorder selected from asthma, chronic obstructive pulmonary disease (COPD), exacerbation of airways hyper reactivity consequent to other drug therapy, allergic rhinitis, sinusitis, pulmonary vasoconstriction, inflammation, allergies, impeded respiration, respiratory distress syndrome, pulmonary hypertension, pulmonary vasoconstriction, and any other respiratory disease, condition, trait, genotype or phenotype that can respond to the administration of, for example, a LAMA, LABA, SABA, ICS, non-corticosteroid anti-inflammatory agent, or other active agent as described herein, whether alone or in combination with other therapies. In certain embodiments, the compositions, systems and methods described herein can be used to treat pulmonary inflammation and obstruction associated with cystic fibrosis. In specific embodiments of methods for treating patients suffering from an inflammatory or obstructive pulmonary disease or condition, the pulmonary disease of condition is selected from those specifically described herein, and the method includes pulmonary delivery of a composition according to the present description to the patient via an MDI, wherein the pulmonary delivery of such composition includes administering one or more active agents at a dose or dose range as described in association with the compositions disclosed herein.

### Metered Dose Inhaler Systems

As described in relation to the methods provided herein, the compositions disclosed herein may be used in an MDI system. MDIs are configured to deliver a specific amount of a medicament in aerosol form. In one embodiment, an MDI system includes a pressurized, liquid phase formulation-filled canister disposed in an actuator formed with a mouthpiece. The MDI system may include the formulations described herein, which include a suspension medium comprising an HFC propellant (e.g., HFC-152a), at least one species of active agent particles and at least one species of suspending particles. The canister used in the MDI be any of any suitable configuration, and in one exemplary embodiment, the canister may have a volume ranging from about 5 ml to about 25 ml, such as, for example a canister having a 19 ml volume. After shaking the device, the mouthpiece is inserted into a patient's mouth between the lips and teeth. The patient typically exhales deeply to empty the lungs and then takes a slow deep breath while actuating the cartridge.

Inside an exemplary cartridge is a metering valve including a metering chamber capable of holding a defined volume of the formulation (e.g., 63 μl or any other suitable volume available in commercially available metering valves), which is released into an expansion chamber at the distal end of the valve stem when actuated. The actuator retains the canister and may also include a port with an actuator nozzle for receiving the valve stem of the metering valve. When actuated, the specified volume of formulation travels to the expansion chamber, out the actuator nozzle and into a high-velocity spray that is drawn into the lungs of a patient.

The following abbreviations are used throughout the present disclosure including the Drawing and Examples:
AB: Albuterol
AS: Albuterol Sulfate
BD: Budesonide
FF: Formoterol Fumarate
GP: Glycopyrrolate
RF: Roflumilast
BGF: budesonide/glycopyrrolate/formoterol (combo)
GFF: glycopyrolate/formoterol fumarate (combo)
BDA-152a: budesonide/albuterol (combo) in HFC-152a
BFF-152a: budesonide/formoterol fumarate (combo) in HFC-152a
BGF-152a: budesonide/glycopyrrolate/formoterol (combo) in HFC-152a
GFF-152a: glycopyrrolate/formoterol fumarate (combo) in HFC-152a
BGFR: budesonide/glycopyrrolate/formoterol fumarate/roflumilast (combo)
CFC-11: Trichlorofluoromethane
CFC-113: 1,1,2-Trichloro-1,2,2-trifluoroethane
CFC-114: 1,2-Dichlorotetrafluoroethane
HCFC-124: 1-Chloro-1,2,2,2-tetrafluoroethane
HFA-227ea: 1,1,1,2,3,3,3-Heptafluoropropane
HFC-125: Pentafluoroethane, also known as 1,1,1,2,2-Pentafluoroethane
HFC-152a: 1,1-Difluoroethane
HFC-245cb: 1,1,1,2,2-Pentafluoropropane
HFO-1225ye(Z): cis-1,2,3,3,3-Pentafluoropropene
HFO-1225ye(E): trans-1,2,3,3,3-Pentafluoropropene
HFO-1234yf: 2,3,3,3-Tetrafluoropropene
HFO-1234ze(Z): cis-1,3,3,3-Tetrafluoroprop-1-ene

The specific examples included herein are for illustrative purposes only and are not to be considered as limiting to this disclosure. Moreover, the compositions, systems and methods disclosed herein have been described in relation to certain embodiments thereof, and many details have been set forth for purposes of illustration, it will be apparent to those skilled in the art that the disclosure is susceptible to additional embodiments and that certain of the details described herein may be varied without departing from the basic principles of the disclosure. Any active agents and reagents used in the following examples are either commercially available or, with the benefit of the teachings provided herein, can be prepared according to standard literature procedures by those skilled in the art. The entire contents of all publications, patents, and patent applications referenced herein are hereby incorporated herein by reference.

### EXAMPLES

### Example 1

Suspending particles were manufactured by spray drying an emulsion of PFOB (perfluorooctyl bromide) and water stabilized by DSPC (1,2-Distearoyl-sn-Glycero-3-Phosphocholine). Detailed preparation procedures have been documented previously. The particle size distribution of the suspending particles was determined by laser diffraction. 50% by volume of the suspending particles were smaller than 2.9 μm, the Geometric Standard Deviation of the distribution was 1.8.

Active agent particles formed of glycopyrrolate (Pyrrolidinium, 3-((cyclopentylhydroxyphenylacetyl)oxy)-1,1-dimethyl-, bromide) were formed by micronizing glycopyrrolate using a jet mill. The particle size distribution of the micronized glycopyrrolate (GP) was determined by laser diffraction. 50% by volume of the micronized particles exhibited an optical diameter smaller than 2.1 μm, 90% by volume were smaller than 5 μm.

Formoterol fumarate, (±)-2-hydroxy-5-[(1RS)-1-hydroxy-2-[[(1RS)-2-(4-methoxyphenyl)-1-methylethyl]-amino]ethyl]formanilide fumarate, also known as (±)-2′-hydroxy-5-[(RS)-1-hydroxy-2-[[RS)-p-methoxy-α-methylphenethyl]-amine]ethyl]formanilide fumarate, dihydrate was received micronized by the manufacturer (Inke) and used as active agent particles. The particle size distribution of the micronized formoterol fumarate (FF) was determined by laser diffraction. 50% by volume of the micronized particles exhibited an optical diameter smaller than 1.6 μm, and 90% by volume exhibited an optical diameter smaller than 3.9 μm.

Active agent particles formed of budesonide, 16,17-(butylidenebis(oxy))-11,21-dihydroxy-, (11-β,16-α)-pregna-1,4-diene-3,20-dione, were formed by micronizing budesonide using a jet mill. The particle size distribution of the budesonide (BD) was determined by laser diffraction. 50% by volume of the micronized particles exhibited an optical diameter smaller than 1.9 μm, 90% by volume exhibited an optical diameter smaller than 4.3 μm.

Active agent particles formed of albuterol sulfate, α1 [(tert-butylamino) methyl]-4-hydroxy-m xylene-α,α′-diol sulfate, were formed by micronizing albuterol sulfate using a jet mill. The particle size distribution of the albuterol sulfate (AS) was determined by laser diffraction. 50% by volume of the micronized particles exhibited an optical diameter smaller than 1.5 μm, 90% by volume exhibited an optical diameter smaller than 3.3 μm.

Metered dose inhalers were prepared by first dispensing the appropriate quantities of suspending particles and active agent particles an addition vessel (AV) and adding an appropriate quantity of HFC-152a (1,1-Difluoroethane) propellant. The mixture is agitated to facilitate powder wetting and then transferred to a pressure vessel where the suspension is mixed. Valves comprised of 50 uL metering chambers (BK357, Bespak, King's Lynn, UK) are crimped onto fluorinated ethylene polymer (FEP) coated aluminum cans (Presspart, Blackburn, UK) and the suspension is then pressure filled through the valve. The canisters were fitted with polypropylene actuators with a 0.32 mm or 0.39 mm orifice (#10024269, Bespak, King's Lynn, UK).

### Example 2

Metered dose inhalers containing a triple co-suspension composition comprising glycopyrrolate, budesonide, and formoterol active agent particles were prepared, with each type of active agent particle being provided as a micronized, crystalline API material. The active agent particles were suspended in HFC-152a propellant with phospholipid particles. As shown in FIG. 1 and Table 1, the three types of active agent particles containing phospholipid particles showed uniform aerodynamic particle size deposition profiles.

TABLE 1
BD, GP, and FF Fine Particle Fraction (FPF), Fine Particle
Dose (FPD), Mass Median Aerodynamic Diameter (MMAD),
and Throat Deposition of BGF-152a

| | FPF, <6.4 | FPD, <6.4 | |
|---|---|---|---|
| Active | μm (%) | μm (μg/act) | MMAD (μm) |
| BD | 48 ± 3 | 80.92 ± 8.32 | 3.87 ± 0.14 |
| GP | 49 ± 3 | 3.69 ± 0.36 | 3.70 ± 0.16 |
| FF | 50 ± 3 | 2.47 ± 0.27 | 3.69 ± 0.15 |

### Example 3

The fine particle fraction (FPF) present in the delivered dose upon actuation of an MDI containing budesonide, formoterol, or glycopyrrolate active agent particles and phospholipid particles was measured following storage of the MDI under various temperature and relative humidity conditions for varied periods of time. (See, Tables 2 and 3 below)

The fine particle mass (FPM) present in the delivered dose upon actuation of an MDI containing budesonide and phospholipid particles was measured following storage of the MDI under various temperature and relative humidity conditions for varied periods of time. (See, Tables 2 and 3 below)

TABLE 2
BD, GP, and FF Fine Particle Fraction (FPF), Fine Particle
Dose (FPD), Mass Median Aerodynamic Diameter (MMAD),
and Throat Deposition Stability Data for BGF-152a

| | | FPF, <6.4 | FPD, <6.4 | MMAD |
|---|---|---|---|---|
| Active | Storage | μm (%) | μm (μg/act) | (μm) |
| BD | Initial | 46 ± 2 | 68.00 ± 3.08 | 4.02 ± 0.07 |
| | 1 month | 44 ± 1 | 65.60 ± 2.07 | 3.93 ± 0.03 |
| | 25° C./60% RH | | | |
| | 3 month | 49 ± 1 | 73.40 ± 1.67 | 3.82 ± 0.10 |
| | 25° C./60% RH | | | |
| | 6 month | 49 ± 1 | 74.80 ± 1.30 | 3.92 ± 0.04 |
| | 25° C./60% RH | | | |
| | 12 month | 48 ± <1 | 73.20 ± 1.10 | 3.89 ± 0.05 |
| | 25° C. /60% RH | | | |
| GP | Initial | 47 ± 2 | 3.18 ± 0.16 | 3.90 ± 0.07 |
| | 1 month | 45 ± 1 | 3.02 ± 0.08 | 3.81 ± 0.04 |
| | 25° C./60% RH | | | |
| | 3 month | 49 ± 1 | 3.42 ± 0.08 | 3.68 ± 0.10 |
| | 25° C./60% RH | | | |
| | 6 month | 50 ± 1 | 3.50 ± 0.07 | 3.78 ± 0.05 |
| | 25° C./60% RH | | | |
| | 12 month | 48 ± 2 | 3.46 ± 0.05 | 3.75 ± 0.05 |
| | 25° C. /60% RH | | | |
| FF | Initial | 47 ± 2 | 2.06 ± 0.11 | 3.83 ± 0.09 |
| | 1 month | 45 ± 1 | 1.96 ± 0.05 | 3.72 ± 0.04 |
| | 25° C./60% RH | | | |
| | 3 month | 50 ± 0 | 2.20 ± 0.07 | 3.56 ± 0.12 |
| | 25° C./60% RH | | | |
| | 6 month | 51 ± 1 | 2.26 ± 0.05 | 3.62 ± 0.04 |
| | 25° C./60% RH | | | |
| | 12 month | 50 ± 1 | 2.16 ± 0.05 | 3.59 ± 0.06 |
| | 25° C. /60% RH | | | |

TABLE 3
BD, GP, and FF Fine Particle Fraction (FPF), Fine Particle
Dose (FPD), Mass Median Aerodynamic Diameter (MMAD),
and Throat Deposition Stability Data for BGF-152a

| | | FPF, <6.4 | FPD, <6.4 | MMAD |
|---|---|---|---|---|
| Active | Storage | μm (%) | μm (μg/act) | (μm) |
| BD | Initial | 46 ± 2 | 68.00 ± 3.08 | 4.02 ± 0.07 |
| | 1 month | 46 ± 1 | 68.40 ± 2.19 | 3.95 ± 0.04 |
| | 40° C./75% RH | | | |
| | 3 month | 49 ± 1 | 77.40 ± 1.82 | 3.88 ± 0.02 |
| | 40° C./75% RH | | | |
| | 6 month | 48 ± 2 | 74.20 ± 6.30 | 4.05 ± 0.03 |
| | 40° C./75% RH | | | |
| GP | Initial | 47 ± 2 | 3.18 ± 0.16 | 3.90 ± 0.07 |
| | 1 month | 46 ± 1 | 3.18 ± 0.15 | 3.83 ± 0.06 |
| | 40° C./75% RH | | | |
| | 3 month | 50 ± 1 | 3.62 ± 0.08 | 3.73 ± 0.02 |
| | 40° C./75% RH | | | |
| | 6 month | 49 ± 2 | 3.44 ± 0.27 | 3.92 ± 0.03 |
| | 40° C./75% RH | | | |
| FF | Initial | 47 ± 2 | 2.06 ± 0.11 | 3.83 ± 0.09 |
| | 1 month | 47 ± 1 | 2.00 ± 0.07 | 3.72 ± 0.05 |
| | 40° C./75% RH | | | |
| | 3 month | 50 ± 1 | 2.18 ± 0.04 | 3.62 ± 0.01 |
| | 40° C./75% RH | | | |
| | 6 month | 50 ± 2 | 2.00 ± 0.20 | 3.80 ± 0.04 |
| | 40° C./75% RH | | | |

### Example 4

Delivered dose uniformity upon actuation of an MDI containing budesonide, glycopyrrolate, and formoterol active agent particles and phospholipid particles was measured following storage of the MI under various temperature and relative humidity conditions for varied periods of time. (See, FIGS. 8 to 13)

### Example 5

Degradation of budesonide, glycopyrrolate, formoterol active agent particles in an MDI canister containing active agent particles and phospholipid particles was measured following storage of the MI under various temperature and relative humidity conditions for varied periods of time. (See, Tables 4 and 5 below)

TABLE 4
BD, GP, and FF Related Substance Stability Data for
BGF-152a, 25° C./60% RH - Valve Down, Protected

| | | Time (months) | | | | |
|---|---|---|---|---|---|---|
| | Initial | 1 | 3 | 6 | 9 | 12 |
| Budesonide related | 0.15 | 0.15 | 0.15 | 0.16 | 0.16 | 0.23 |
| substances (% w/w | | | | | | |
| mean) | | | | | | |
| Glycopyrronium | <0.10 | <0.10 | <0.10 | <0.10 | <0.10 | <0.10 |
| related substances | | | | | | |
| (% w/w mean) | | | | | | |
| Formoterol fumarate | <0.10 | <0.10 | 0.16 | 0.41 | 0.64 | 1.07 |
| related substances | | | | | | |
| (% w/w mean) | | | | | | |

TABLE 5
BD, GP, and FF Related Substance Stability Data
for BGF-152a, 40° C./75% RH-Valve Down, Protected

| | | Time (months) | | |
|---|---|---|---|---|
| | Initial | 1 | 3 | 6 |
| Budesonide related | 0.15 | 0.15 | 0.26 | 0.29 |
| substances (% w/w mean) | | | | |
| Glycopyrronium related | <0.10 | <0.10 | <0.10 | <0.10 |
| substances (% w/w mean) | | | | |
| Formoterol fumarate related | <0.10 | 0.76 | 3.07 | 5.62 |
| substances (% w/w mean) | | | | |

### Example 6

A randomized, single blind, 3-period, 3-treatment, single-dose, crossover study was conducted to assess the relative bioavailability of BGF MDI HFC-152a and BGF MDI HFO-1234ze compared with BGF MDI HFA-134a in healthy subjects.

The investigational medical products include (1) Test Product of budesonide/glycopyrronium/formoterol (BGF) metered dose inhaler (MDI) formulated with HFC-152a propellant and (2) Reference Product of budesonide/glycopyrronium/formoterol (BGF) metered dose inhaler (MDI) formulated with HFA-134a propellant. The indication studied is chronic obstructive pulmonary disease (COPD) and the development phase is Phase 1.

### Study Objectives:

### Primary Objective:

To evaluate the relative bioavailabilities between the test formulations and the reference formulation for fixed dose combinations (FDCs) of budesonide, glycopyrronium, and formoterol when administered as budesonide, glycopyrronium, and formoterol (BGF) metered dose inhaler (MDI) with 3 different propellants.

### Secondary Objectives:

To determine the pharmacokinetic (PK) parameters of BGF when administered as 3 different propellant formulations. To assess the safety and tolerability of a combination of BGF when administered as single doses in 3 different propellant formulations in healthy subjects.

### Study Design:

This study was a randomized, single blind, 3-period, 3-treatment, single-dose, single-center, crossover study. The study included the assessment of PK properties of BGF MDI formulated with 3 different propellants: hydrofluoroolefin (HFO-1234ze) —Treatment A (test), hydrofluorocarbon (HFC-152a) —Treatment B (test), and hydrofluoroalkane (HFA-134a) —Treatment C (reference).

The study comprised of:
Screening period: up to 28 days prior to first dosing.
Three treatment periods of maximum 3 days each: subjects were resident from the morning of the day before the first dosing with BGF MDI (Day −1) in Treatment Period 1, throughout all treatment and washout periods up to discharge on Day 2 of Treatment Period 3.
Follow-up: within 3 to 7 days after the last administration of BGF MDI. There was a washout period of 3 to 7 days between each dose. Each subject received 3 single-dose treatments of BGF MDI (1 dose HFO-1234ze [Treatment A]; 1 dose HFC-152a [Treatment B] and 1 dose HFA-134a [Treatment C]), following an overnight fast of at least 8 hours.

### Main Inclusion Criteria:

Healthy, non-smoking male subjects aged 18 to 60 years with suitable veins for cannulation or repeated venepuncture. Subjects had to have a body mass index (BMI) between 18 and 30 kg/m2, inclusive and weigh at least 50 kg and no more than 100 kg, inclusive. Subjects had to have a forced expiratory volume in one second (FEV1) ≥80% of the predicted value regarding age, height, and ethnicity at the screening visit.

### Investigational Medicinal Products:

Treatment A (test): BGF MDI HFO-1234ze(E) with strength/concentrations of 160/7.2/4.8 μg per actuation.

Treatment B (test): BGF MDI HFC-152a with strength/concentrations of 160/7.2/4.8 μg per actuation.

Treatment C (reference): BGF MDI HFA-134a with strength/concentrations of 160/7.2/4.8 μg per actuation.

### Duration of Study:

Each subject was to be involved in the study for up to 53 days.

### Treatment Compliance:

Dosing took place at the Parexel Early Phase Clinical Unit in Los Angeles. The administration of all investigational medicinal products (IMPs) was recorded in Parexel's electronic source data capturing and information management system (CLINBASE™). Compliance was assured by direct supervision and witnessing of IMP administration.

### Criteria for Evaluation:

### Pharmacokinetic Parameters:

Primary PK parameters: Cmax, AUCinf, and AUClast for test and reference treatment.
Secondary PK parameters: tmax, t_{1/2}λz, MRT, λz, CL/F, Vz/F, TRCmax, TRAUCinf, and TRAUClast.

### Safety Variables:

Adverse events (AEs)/Serious adverse events (SAEs).
Vital signs (systolic and diastolic blood pressure, pulse rate, body temperature, oxygen saturation, and respiratory rate).
Twelve-lead safety and digital electrocardiograms (ECGs) as well as cardiac telemetry
Physical examination.
Laboratory assessments (hematology, clinical chemistry and urinalysis)
Spirometry.
Taste assessment.

### Statistical Methods:

### Determination of Sample Size:

This was a pilot PK study to determine the relative bioavailabilities between 2 test formulations of BGF MDI compared with the conventional formulation. Therefore, no sample size calculation was performed.

It was expected that 48 healthy subjects (number of subjects were increased from 24 to 48 as per protocol amendment 2 to account for replacement subjects due to a dosing deviation involving the first 23 subjects) were to be randomized to a 6 sequence Williams design for 3 periods and 3 treatments: ABC, BCA, CAB, ACB, BAC and CBA, in order to ensure at least 20 evaluable subjects at the end of the last treatment period.

Subjects were considered evaluable if they had an evaluable PK profile, i.e., (1) received active treatment, (2) did not significantly violate protocol inclusion or exclusion criteria, or deviate significantly from the protocol, and (3) did not have unavailable or incomplete data which may have influenced the PK analysis. Presentation and Analysis of Pharmacokinetic Data:

All PK concentrations, parameter summaries and statistical analyses were presented for the PK Analysis Set, unless otherwise specified. The PK concentration and parameter listings were presented for the Safety Analysis Set and included all reportable individual PK results. Individual PK concentration and parameter data for any subjects not included in the PK Analysis Set or excluded from the descriptive summary tables, figures and/or inferential statistical analyses were included in the listings and flagged with an appropriate footnote.

The test treatments, Treatments A and B (BGF MDI HFO and BGF MDI HFC, respectively), were separately compared to the reference treatment, Treatment C (BGF MDI HFA), for each analyte. The statistical analyses were performed using a linear mixed effects analysis of variance model, using the natural logarithm of Cmax, AUCinf, and AUClast as the response variables, with sequence, and period, treatment as fixed effects and subject nested within sequence as random effect. Transformed back from the logarithmic scale, geometric means together with the intra-subject coefficient of variation confidence intervals (CIs) (2-sided 95%) for Cmax, AUCinf, and AUClast were estimated and presented. In addition, ratios of geometric means together with CIs (2-sided 90%) were estimated and presented.

Additionally, the median difference in untransformed tmax between the test treatments and the reference treatment for each analyte and the corresponding 90% CIs for the median differences, for each analyte were calculated using the non parametric Hodges Lehmann method.

### Presentation and Analysis of Safety and Eligibility Data:

Safety data (scheduled and unscheduled) were presented in the data listings. Continuous variables were summarized using descriptive statistics (n, mean, standard deviation [SD], minimum, median, maximum) by treatment. Categorical variables were summarized in frequency tables (frequency and proportion) by treatment, if applicable. The analysis of the safety variables was based on the Safety Analysis Set.

Adverse events were summarized by Preferred Term (PT) and System Organ Class (SOC) using Medical Dictionary for Regulatory Activities (MedDRA) vocabulary. Furthermore, listings of SAEs and AEs that led to withdrawal were made and the number of subjects who had any AEs, SAEs, AEs that led to withdrawal, and AEs with severe intensity were summarized. Adverse events that occurred before dosing were reported separately.

Tabulations and listings of data for vital signs, clinical laboratory tests, digital ECGs, and 12-lead safety ECGs (listings only), telemetry (listings only), and spirometry were presented. Results from the taste assessment were presented separately in listings only. Any new or aggravated clinically relevant abnormal medical physical examination finding compared to the baseline assessment was reported as an AE. Data were summarized for the observed values at each scheduled assessment, together with the corresponding changes from the baseline when baseline was defined. Clinical laboratory data were reported in the units provided by the clinical laboratory for the Safety Review Committee (SRC) meeting, and in Systeme International (SI) units in the Clinical Study Report (CSR).

Out of range values for safety laboratory assessments were flagged in individual listings as well as summarized descriptively using agreed standard reference ranges and/or extended reference ranges (eg, AstraZeneca, program, or laboratory ranges).

### Protocol Deviations:

In total, important protocol deviations were reported for 26 (55.3%) subjects during the study:
For Treatment A (HFO propellant): 23 (48.9%) subjects were reported with other important protocol deviations (subject did not self dose with the inhaler as outlined in the protocol. Nurse administered the dose).
For Treatment B (HFC propellant): 23 (48.9%) subjects were reported with other important protocol deviations (subject did not self dose with the inhaler as outlined in the protocol. Nurse administered the dose) and 2 (4.3%) subjects did not receive the full dose expected due to issues during inhalation.
For Treatment C (HFA propellant): 23 (48.9%) subjects were reported with other important protocol deviations (subject did not self dose with the inhaler as outlined in the protocol. Nurse administered the dose) and 1 (2.1%) subject did not receive the full dose expected due to issues during inhalation.

The number of subjects were increased from 24 to 48 as per protocol amendment 2 to account for replacement subjects due to a dosing deviation involving the first 23 subjects.

There were 23 subjects excluded from the PK Analysis Set due to protocol deviations reported. No important protocol deviations related to COVID-19 were reported during the study.

### Pharmacokinetic Results:

Systemic exposure to budesonide from BGF MDI HFC was comparable to BGF MDI HFA, with GMRs and 90% CIs of 98.78% (78.67%, 124.0%), 98.03% (83.33%, 115.3%) and 98.80% (84.59%, 115.4%) for Cmax, AUCinf and AUClast, respectively.
Systemic exposure to glycopyrronium from BGF MDI HFC was comparable to BGF MDI HFA, with GMRs and 90% CIs of 94.88% (74.69%, 120.5%) and 99.71% (80.84%, 123.0%) for Cmax and AUClast, respectively.
Systemic exposure to formoterol from BGF MDI HFC was comparable to BGF MDI HFA, with GMRs and 90% CIs of 100.1% (83.78%, 119.5%), 116.7% (86.31%, 157.8%) and 107.0% (88.82%, 128.9%) for Cmax, AUCinf and AUClast, respectively.

### Safety Results:

There were no deaths, SAEs, or AEs that led to the discontinuation of the IMP reported during this study.
No new safety signals were observed, no clinically relevant trends were observed for vital signs, physical examination, laboratory results, spirometry and taste assessment, and no abnormal clinically significant 12-lead safety and digital ECG, as well as cardiac telemetry findings were reported.
The combination of budesonide, glycopyrronium, and formoterol when administered as single doses in 3 different propellant formulations demonstrated an acceptable safety profile and was well tolerated in the studied population.

In view of this clinical study, systemic exposure to budesonide, glycopyrronium, and formoterol was similar for BGF MDI HFC-152a compared with the reference product, BGF MDI HFA-134a. There was no indication of meaningful differences between the products in this taste assessment. The combination of budesonide, glycopyrronium, and formoterol when administered as single doses in HFC-152a and HFA-134a formulations demonstrated an acceptable safety profile and was well tolerated in the studied population.

### Example 7

Metered dose inhalers containing a dual co-suspension composition comprising glycopyrrolate and formoterol fumarate active agent particles were prepared, with each type of active agent particle being provided as a micronized, crystalline API material. The active agent particles were suspended in HFC-152a propellant with phospholipid particles. As shown in FIG. 14 and Table 6, the two types of active agent particles containing phospholipid particles showed uniform aerodynamic particle size deposition profiles. (See, Table 6 below)

TABLE 6
GP and FF Fine Particle Fraction (FPF), Fine Particle Dose
(FPD) and Mass Median Aerodynamic Diameter
(MMAD) of GFF-152a

| | FPF, <6.4 | FPD, <6.4 | MMAD |
|---|---|---|---|
| Active | μm (%) | μm (μg/act) | (μm) |
| GP | 48 ± 1 | 3.56 ± 0.18 | 3.43 ± 0.07 |
| FF | 47 ± 1 | 2.18 ± 0.08 | 3.50 ± 0.05 |

### Example 8

Metered dose inhalers containing a dual co-suspension composition comprising budesonide and albuterol sulfate active agent particles were prepared, with each type of active agent particle being provided as a micronized, crystalline API material. The active agent particles were suspended in HFC-152a propellant with phospholipid particles. As shown in FIG. 16 and Table 7, the two types of active agent particles containing phospholipid particles showed uniform aerodynamic particle size deposition profiles. (See, Table 7 below)

TABLE 7
BD and AB Fine Particle Fraction (FPF), Fine Particle
Dose (FPD) and Mass Median Aerodynamic
Diameter (MMAD) of BDA-152a

| | FPF, <6.4 | FPD, <6.4 | MMAD |
|---|---|---|---|
| Active | μm (%) | μm (μg/act) | (μm) |
| BD | 30 ± 1 | 24.9 ± 0.55 | 4.63 ± 0.05 |
| AB | 31 ± 1 | 28.68 ± 0.61 | 4.36 ± 0.06 |

### Example 9

Delivered dose uniformity upon actuation of an MDI containing glycopyrrolate and formoterol fumarate active agent particles and phospholipid particles was measured after preparation of the inhaler. The data demonstrates a similar delivery of glycopyrrolate and formoterol fumarate at both near the beginning and near the end of the inhaler life. (See, FIG. 15)

### Example 10

Delivered dose uniformity upon actuation of an MDI containing budesonide and albuterol sulfate active agent particles and phospholipid particles was measured after preparation of the inhaler. The data demonstrates a similar delivery of budesonide and albuterol sulfate at both near the beginning and near the end of the inhaler life. (See, FIG. 17)

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, unless specified otherwise herein. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A pharmaceutical composition deliverable from a metered dose inhaler, the pharmaceutical composition comprising:
a propellant of pharmaceutical grade 1,1-Difluoroethane (HFC-152a);
a plurality of active agent particles; and
a plurality of phospholipid particles comprising perforated microstructures;
wherein the active agent particles comprise an active agent selected from a long-acting muscarinic antagonist (LAMA), a long-acting 2-agonist (LABA), a short-acting beta-agonist (SABA), an inhaled corticosteroid (ICS), and a non-corticosteroid anti-inflammatory agent.

2. The pharmaceutical composition according to claim 1, wherein the plurality of active agent particles comprises two or more species of active agent particles, wherein each species of active agent particle comprises a different active agent selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent.

3. A pharmaceutical composition deliverable from a metered dose inhaler, the pharmaceutical composition comprising:
a propellant of pharmaceutical grade 1,1-Difluoroethane (HFC-152a);
a plurality of a first species of active agent particle;
a plurality of a second species of active agent particle; and
a plurality of phospholipid particles comprising perforated microstructures;
wherein the first species of active agent particles comprise a first active agent and the second species of active agent particles comprise a second active agent, and wherein the first and second active agents are selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent.

4. The pharmaceutical composition according to claim 3, further comprising a plurality of a third species of active agent particle; wherein the third species of active agent particles comprise a third active agent selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent.

5. The pharmaceutical composition according to claim 4, further comprising a plurality of a fourth species of active agent particle; wherein the fourth species of active agent particles comprise a fourth active agent selected from a long-acting muscarinic antagonist (LAMA), a long-acting β2-agonist (LABA), a short-acting beta-agonist (SABA), an inhaled corticosteroid (ICS), and a non-corticosteroid anti-inflammatory agent.

6. The pharmaceutical composition according to any one of claims 1 to 5, wherein the LAMA is present at a concentration in the range of about 0.04 mg/mL to about 2.25 mg/mL.

7. The pharmaceutical composition according to any one of claims 1 to 5, wherein the LABA is present at a concentration in the range of about 0.01 mg/mL to about 1 mg/mL.

8. The pharmaceutical composition according to any one of claims 1 to 5, wherein the ICS is present at a concentration in the range of about 0.1 mg/mL to about 20 mg/mL.

9. The pharmaceutical composition according to any one of claims 1 to 5, wherein the non-corticosteroid anti-inflammatory agent is present at a concentration in the range of about 0.1 mg/mL to about 20 mg/mL.

10. The pharmaceutical composition according to any one of claims 1 to 9, wherein the phospholipid particles are present at a concentration in the range of about 0.1 mg/mL to about 10 mg/mL.

11. The pharmaceutical composition according to any one of claims 1 to 10, wherein the perforated microstructures comprise 1,2-Distearoyl-sn-glycero-3-phosphocholine (DSPC) and calcium chloride.

12. The pharmaceutical composition according to any one of claims 1 to 11, wherein the phospholipid particles exhibit a volume median optical diameter selected from between about 0.2 μm and about 50 μm, between about 0.5 μm and about 15 μm, between about 1.5 μm and about 10 μm, and between about 2 μm and about 5 μm.

13. The pharmaceutical composition according to any one of claims 1 to 12 wherein a total mass of the phospholipid particles exceeds a total mass of:
i) the plurality of active agent particles of claim 1;
ii) any one of the first, second, third, or fourth species of active agent particles; or
iii) the combination of any two of the first, second, third, and fourth species of active agent particles.

14. The pharmaceutical composition according to any one of claims 3 to 13 wherein the first active agent is a LAMA; and the second active agent is a LABA.

15. The pharmaceutical composition according to any one of claims 4 to 13, wherein the first active agent is a LAMA; the second active agent is a LABA; and the third active agent is an ICS.

16. The pharmaceutical composition according to any one of claims 5 to 13, wherein the first active agent is a LAMA; the second active agent is a LABA; the third active agent is an ICS; and the fourth active agent is a non-corticosteroid anti-inflammatory agent.

17. The pharmaceutical composition according to any one of claims 3 to 13, wherein the first active agent is a SABA; and the second active agent is an ICS.

18. The pharmaceutical composition according to any one of claims 3 to 13, wherein the first active agent is a LABA; and the second active agent is an ICS.

19. The pharmaceutical composition according to any one of the preceding claims, wherein the LAMA is selected from glycopyrrolate, dexpirronium, tiotropium, trospium, aclidinium, umeclidinium, and darotropium; or a pharmaceutically acceptable salt or solvate thereof.

20. The pharmaceutical composition according to any one of the preceding claims, wherein the LABA is selected from bambuterol, clenbuterol, formoterol, salmeterol, carmoterol, milveterol, indacaterol, vilanterol, and saligenin- or indole-containing and adamantyl-derived β2 agonists; or a pharmaceutically acceptable salt or solvate thereof.

21. The pharmaceutical composition according to any one of the preceding claims, wherein the SABA is selected from bitolterol, carbuterol, fenoterol, hexoprenaline, isoprenaline (isoproterenol), levosalbutamol, orciprenaline (metaproterenol), pirbuterol, procaterol, rimiterol, albuterol (salbutamol), terbutaline, tulobuterol, reproterol, and epinephrine; or a pharmaceutically acceptable salt or solvate thereof.

22. The pharmaceutical composition according to any one of the preceding claims, wherein the ICS is selected from beclomethasone, budesonide, ciclesonide, flunisolide, fluticasone, methylprednisolone, mometasone, prednisone and triamcinolone; or a pharmaceutically acceptable salt or solvate thereof.

23. The pharmaceutical composition according to any one of the preceding claims, wherein the non-corticosteroid anti-inflammatory agent is roflumilast or a pharmaceutically acceptable salt or solvate thereof.

24. The pharmaceutical composition according to any one of the preceding claims, exhibiting an enhanced robustness in simulated use testing (SUT).

25. The pharmaceutical composition according to any one of the preceding claims, exhibiting less than about 1.0%, 0.5%, 0.4%, 0.3%, 0.2%, or 0.1% weight loss in the metered dose inhaler at 25° C./60% RH per year.

26. The pharmaceutical composition according to any one of the preceding claims, comprising:
a propellant of pharmaceutical grade HFC-152a;
a plurality of glycopyrrolate particles;
a plurality of formoterol particles; and
a plurality of phospholipid particles comprising perforated microstructures.

27. The pharmaceutical composition according to any one of the preceding claims, comprising:
a propellant of pharmaceutical grade HFC-152a;
a plurality of glycopyrrolate particles;
a plurality of formoterol particles;
a plurality of budesonide particles; and
a plurality of phospholipid particles comprising perforated microstructures.

28. The pharmaceutical composition according to any one of the preceding claims, comprising:
a propellant of pharmaceutical grade HFC-152a;
a plurality of albuterol particles;
a plurality of budesonide particles; and
a plurality of phospholipid particles comprising perforated microstructures.

29. The pharmaceutical composition according to any one of the preceding claims, comprising:
a propellant of pharmaceutical grade HFC-152a;
a plurality of formoterol particles;
a plurality of budesonide particles; and
a plurality of phospholipid particles comprising perforated microstructures.

30. The pharmaceutical composition according to any one of the preceding claims, comprising:
a propellant of pharmaceutical grade HFC-152a;
a plurality of glycopyrrolate particles;
a plurality of formoterol particles;
a plurality of budesonide particles;
a plurality of roflumilast particles; and
a plurality of phospholipid particles comprising perforated microstructures.

31. The pharmaceutical composition according to any one of the preceding claims, wherein the glycopyrrolate active agent particles are in the propellant at a concentration sufficient to provide a delivered dose of glycopyrrolate per actuation of the metered dose inhaler selected from between about 5 μg and about 50 μg per actuation, between about 2 μg and about 25 μg per actuation, and between about 6 μg and about 15 μg per actuation.

32. The pharmaceutical composition according to any one of the preceding claims, wherein the concentration of glycopyrrolate in the propellant is between about 0.04 mg/ml and about 2.25 mg/ml.

33. The pharmaceutical composition according to any one of the preceding claims, wherein at least 90% of the glycopyrrolate active agent particles by volume exhibit an optical diameter of 7 μm or less.

34. The pharmaceutical composition according to any one of the preceding claims, wherein the formoterol active agent particles are included in the composition at a concentration sufficient to provide a delivered dose of formoterol selected from between about 1 μg and about 30 μg, between about 0.5 μg and about 10 μg, between about 2 μg and 5 μg, between about 3 μg and about 10 μg, between about 5 μg and about 10 μg, and between 3 μg and about 30 μg per actuation of the metered dose inhaler.

35. The pharmaceutical composition according to any one of the preceding claims, wherein the concentration of formoterol in the propellant is selected from between about 0.01 mg/ml and about 1 mg/ml, between about 0.01 mg/ml and about 0.5 mg/ml, and between about 0.03 mg/ml and about 0.4 mg/ml.

36. The pharmaceutical composition according to any one of the preceding claims, wherein at least 90% of the formoterol active agent particles by volume exhibit an optical diameter of 5 μm or less.

37. The pharmaceutical composition according to any one of the preceding claims, wherein the budesonide active agent particles are included in the composition at a concentration sufficient to provide a delivered dose of budesonide selected from between about 50 μg and about 400 μg, between about 20 μg and about 600 μg, between about 30 μg and 100 μg, between about 50 μg and about 200 μg, and between about 150 μg and about 350 μg per actuation of the metered dose inhaler.

38. The pharmaceutical composition according to any one of the preceding claims, wherein the concentration of budesonide in the propellant is selected from between about 0.1 mg/ml and about 20 mg/ml, between about 0.1 mg/ml and about 5 mg/ml, and between about 0.3 mg/ml and about 6 mg/ml.

39. The pharmaceutical composition according to any one of the preceding claims, wherein at least 90% of the budesonide active agent particles by volume exhibit an optical diameter of 7 μm or less.

40. The pharmaceutical composition according to any one of the preceding claims, wherein the albuterol active agent particles are included in the composition at a concentration sufficient to provide a delivered dose of albuterol selected from between about 10 μg and about 200 μg, between about 20 μg and about 300 μg, between about 30 μg and 150 μg, and between about 50 μg and about 200 μg per actuation of the metered dose inhaler.

41. The pharmaceutical composition according to any one of the preceding claims, wherein the concentration of albuterol in the propellant is selected from between about 0.1 mg/ml and about 10 mg/ml, between about 0.1 mg/ml and about 5 mg/ml, and between about 0.3 mg/ml and about 4 mg/ml.

42. The pharmaceutical composition according to any one of the preceding claims, wherein at least 90% of the albuterol active agent particles by volume exhibit an optical diameter of 5 μm or less.

43. The pharmaceutical composition according to any one of the preceding claims, wherein the roflumilast active agent particles are included in the composition at a concentration sufficient to provide a delivered dose of roflumilast selected from between about 50 μg and about 400 μg, between about 20 μg and about 600 μg, between about 30 μg and 100 μg, between about 50 μg and about 200 μg, and between about 150 μg and about 350 μg per actuation of the metered dose inhaler.

44. The pharmaceutical composition according to any one of the preceding claims, wherein the concentration of roflumilast in the propellant is selected from between about 0.1 mg/ml and about 20 mg/ml, between about 0.1 mg/ml and about 5 mg/ml, and between about 0.3 mg/ml and about 6 mg/ml.

45. The pharmaceutical composition according to any one of the preceding claims, wherein at least 90% of the roflumilast active agent particles by volume exhibit an optical diameter of 5 μm or less.

46. The pharmaceutical composition according to any one of the preceding claims, wherein the glycopyrrolate particles comprise glycopyrrolate or a pharmaceutically acceptable salt thereof.

47. The pharmaceutical composition according to claim 46, wherein the glycopyrrolate or a pharmaceutically acceptable salt thereof is in crystalline and/or micronized form.

48. The pharmaceutical composition according to any one of the preceding claims, wherein the formoterol particles comprise formoterol or a pharmaceutically acceptable salt thereof.

49. The pharmaceutical composition according to claim 48, wherein the formoterol or a pharmaceutically acceptable salt thereof is in crystalline and/or micronized form.

50. The pharmaceutical composition according to any one of the preceding claims, wherein the albuterol particles comprise albuterol or a pharmaceutically acceptable salt thereof.

51. The pharmaceutical composition according to claim 50, wherein the albuterol or a pharmaceutically acceptable salt thereof is in crystalline and/or micronized form.

52. The pharmaceutical composition according to any one of the preceding claims, wherein the budesonide particles comprise budesonide which is in crystalline and/or micronized form.

53. The pharmaceutical composition according to any one of the preceding claims, wherein the roflumilast particles comprise roflumilast or a pharmaceutically acceptable salt thereof.

54. The pharmaceutical composition according to claim 53, wherein the roflumilast or a pharmaceutically acceptable salt thereof is in crystalline and/or micronized form.

55. A metered dose inhaler comprising a canister with an outlet valve including an actuator for dispensing a metered amount of a pharmaceutical composition according to any one of claims 1 through 54, wherein the canister contains the pharmaceutical composition.

56. The metered dose inhaler according to claim 55, which exhibits a delivered dose uniformity (DDU) for the pharmaceutical formulation selected from a DDU of ±20%, or better, a DDU of ±15%, or better, and a DDU of ±10%, or better, throughout emptying of the canister.

57. The metered dose inhaler according to claim 55 or 56, which dispenses the pharmaceutical composition at an initial fine particle fraction and the initial fine particle fraction dispensed from the metered dose inhaler is substantially maintained, such that, throughout emptying of the canister, the fine particle fraction delivered from the metered dose inhaler is maintained within 85% of the initial fine particle fraction.

58. The metered dose inhaler according to any one of claims 55 to 57, wherein the fine particle fraction delivered from the metered dose inhaler is maintained within 95% of the initial fine particle fraction.

59. A method of treating a pulmonary disease or disorder in a patient, comprising administering a pharmaceutical composition according to any one of claims 1 to 54 to the patient by actuating a metered dose inhaler; wherein the metered dose inhaler contains the pharmaceutical composition.

60. The method of claim 59, wherein the pulmonary disease or disorder is selected from at least one of asthma, chronic obstructive pulmonary disease (COPD), allergic rhinitis, sinusitis, pulmonary vasoconstriction, inflammation, allergies, impeded respiration, respiratory distress syndrome, pulmonary hypertension, pulmonary inflammation associated with cystic fibrosis, and pulmonary obstruction associated with cystic fibrosis.

61. The method of claim 59 or 60, wherein the pulmonary disease or disorder is asthma or COPD.

62. The method of any one of claims 59 to 61, wherein the metered dose inhaler is described according to any one of the claims 54 to 63.

63. The pharmaceutical composition according to any one of claims 1 to 54 for use in the manufacture of a medicament for the treatment of a pulmonary disease or disorder.

64. The pharmaceutical composition according to any one of claims 1 to 54 for use in the treatment of a pulmonary disease or disorder.

65. The pharmaceutical composition according to any one of claims 1 to 54, which exhibits Cmax, AUCinf or AUClast of any one or more of the active agents, which is 80% to 125% of Cmax, AUCinf or AUClast of the one or more of the active agents of a reference pharmaceutical composition.

66. The metered dose inhaler according to any one of claims 55 to 58, wherein the pharmaceutical composition exhibits Cmax, AUCinf or AUClast of any one or more of the active agents, which is 80% to 125% of Cmax, AUCinf or AUClast of the one or more of the active agents of a reference pharmaceutical composition.

67. The method according to any one of claims 59 to 62, wherein the pharmaceutical composition exhibits Cmax, AUCinf or AUClast of any one or more of the active agents, which is 80% to 125% of Cmax, AUCinf or AUClast of the one or more of the active agents of a reference pharmaceutical composition.
